(19) United States
(12) Patent Application Publication
(10) Pub. No.: US 2018/0362951 A1
(43) Pub. Date: Dec. 20, 2018

(54) Re-targeted Toxin Conjugates

(71) Applicant: Ipsen Bioinnovation Limited, Abingdon (GB)

(72) Inventors: Keith Foster, Salisbury (GB); John Chaddock, Salisbury (GB); Charles Penn, Salisbury (GB)

(21) Appl. No.: 16/021,540

(22) Filed: Jun. 28, 2018

Related U.S. Application Data
(63) Continuation of application No. 13/528,762, filed on Jun. 20, 2012.
Application No. 13/528,762 is a continuation of application No. 12/534,740, filed on Aug. 3, 2009, now abandoned.
Application No. 12/534,740 is a continuation of application No. 10/571,515, filed on Sep. 7, 2006, now abandoned.

(30) Foreign Application Priority Data
Sep. 11, 2003 (GB) 0321344.4

(51) Int. Cl. C12N 9/52; A61K 47/64

(52) U.S. Cl. CPC C12N 9/52; A61K 47/64; A61K 47/642

(57) ABSTRACT

The present invention provides a method for designing a re-targeted toxin conjugate for use in treating a medical condition or disease. Also provided, is the use of said conjugates in the manufacture of a medicament for treating medical conditions or diseases. The conjugates include a Targeting Moiety, which directs the conjugate to a desired target cell, and are characterised by a Targeting Moiety that increases exocytic fusion in the target cell. The present invention also provides methods for identifying agonists suitable for use as Targeting Moieties, and methods for preparing conjugates comprising said Targeting Moieties.

This invention relates to a method for designing a re-targeted toxin conjugate for use in treating a medical condition or disease, and to the use of said conjugate in the manufacture of a medicament for treating medical conditions or diseases.

Toxins may be generally divided into two groups according to the type of effect that they have on a target cell. In more detail, the first group of toxins kill their natural target cells, and are therefore known as cytotoxic toxin molecules. This group of toxins is exemplified inter alia by plant toxins such as ricin, and abrin, and by bacterial toxins such as diphtheria toxin, and Pseudomonas exotoxin A. Cytotoxic toxins have attracted much interest in the design of “magic bullets” (eg, immunoconjugates, which comprise a cytotoxic toxin component and an antibody that binds to a specific marker on a target cell) for the treatment of cellular disorders and conditions such as cancer. Cytotoxic toxins typically kill their target cells by inhibiting the cellular process of protein synthesis.

In contrast, the second group of toxins, which are known as non-cytotoxic toxins, do not (as their name confirms) kill their natural target cells. Non-cytotoxic toxins have attracted much less commercial interest than have their cytotoxic counterparts, and exert their effects on a target cell by inhibiting cellular processes other than protein synthesis. As with their cytotoxic counterparts, non-cytotoxic toxins are produced from a variety of sources such as plants, and bacteria.

Bacterial non-cytotoxic toxins are now described in more detail.

Clostridial neurotoxins are proteins that typically have a molecular mass of the order of 150 kDa. They are produced by various species of bacteria, especially of the genus Clostridium, most importantly C. tetani and several strains of C. botulinum, C. butyricum and C. argentinense. There are at present eight different classes of the dostridial neurotoxin, namely: tetanus toxin, and botulinum neurotoxin in its serotypes A, B, C₁, D, E, F and G, and they all share similar structures and modes of action.

Non-cytotoxic toxins are also produced by other bacteria, such as from the genus Nesseria, most importantly from the species N. gonorrhoeae. For example, Neisseria sp. produce the non-cytotoxic toxin IgA protease (see WO99/58571).

Clostridlal neurotoxins represent a major group of non-cytotoxic toxin molecules, and are synthesised by the host bacterium as single polypeptides that are modified post-translationally by a proteolytic cleavage event to form two polypeptide chains joined together by a disulphide bond. The two chains are termed the heavy chain (H-chain), which has a molecular mass of approximately 100 kDa, and the light chain (L-chain), which has a molecular mass of approximately 50 kDa.

H-chains have two distinct functions, namely binding (ie. to a target cell), and translocation (ie. across an endosomal membrane). The carboxy-terminal portion (Ht) of a H-chain is involved in the hign affinity, neurospecmc binding of the toxin to cell surface receptors, whereas the amino-terminal portion (H_{N}) of the H-chain is central to the translocation of the toxin into the neuronal cell. These two functions have been extensively studied and characterised, and have been mapped to distinct portions within the H-chain (see, for example, Kurazono et al (1992) J. Biol. Chem. 267, 21, pp. 14721-14729; Poulain et al (1989) Eur. J. Biochem. 185, pp. 197-203; Zhou at al (1995), Biochemistry, 34, pp. 15175-15181; Blaustein et al (1987) FEBS Letts., 226, No. 1, pp. 115-120].

L-chains possess a protease function (zinc-dependent endopeptidase activity) and exhibit a high substrate specificity for vesicle and/or plasma membrane associated proteins involved in the exocytic process. L-chains from different clostridial species or serotypes may hydrolyse different but specific peptide bonds in one of three substrate proteins, namely synaptobrevin, syntaxin or SNAP-25. These substrates are important components of the neurosecretory machinery.

By way of specific example, for botulinum neurotoxin serotype A, the above functions have been mapped to amino acid residues 872-1296 for the He portion, amino acid residues 449-871 for the H_{N} portion, and residues 1-448 for the L-chain [see Lacy, D. B. & Stevens, R. C. (1999). Sequence homology and structural analysis of the clostridial neurotoxins. J. Mol. Biol. 291, 1091-1104].

All three of the above-identified domains (ie. H_{C}, H_{N}, and L) are necessary for the in vivo activity of a native neurotoxin, which neurotoxin may cause prolonged muscular paralysis in an affected individual. Corresponding binding, translocation, and protease functions are necessary for the in vivo activity of other non-cytotoxic, bacterial toxins.

It has been well documented in the art that toxin molecules may be re-targeted to a cell that is not the toxin's natural target cell. When so re-targeted, a toxin is capable of binding to a desired target cell and, following subsequent translocation into the cytosol, is capable of exerting its effect on the target cell.

For example, in the context of non-cytotoxic toxin molecules, it has been well documented that a clostridial neurotoxin may be re-targeted by incorporation of a Targeting Moiety (TM), which is not the natural TM of a clostridial neurotoxin. The described chemical conjugation and recombinant methodologies are now regarded as conventional.

In more detail, the following patent publications, in the name of the present Applicant, describe the preparation of modified bacterial conjugates.

WO94/21300 describes the preparation of modified clostridial neurotoxin molecules that, once translocated into the cytosol of a desired target cell, are capable of regulating Integral Membrane Protein (IMP) density present at the cell surface of the target cell. The modified neurotoxin molecules are thus capable of controlling cell activity (eg. glucose uptake) of the target cell.

WO96/33273 describes the preparation of modified clostridial neurotoxin molecules that target peripheral sensory afferents. Once delivered into the cytosol of a peripheral sensory afferent, the modified neurotoxin molecules are capable of demonstrating an analgesic effect.

WO98/07864 describes the preparation of single chain, modified clostridial neurotoxin molecules, which single chain molecules are substantially inactive in terms of sequential binding, translocation and L-chain dependent endopeptidase activities. The single chain molecules are activatable into active di-chain molecules through a proteolytic cleavage reaction.

WO99/17806 describes the preparation of modified dostridial neurotoxin molecules that target primary sensory afferents, which modified neurotoxins are capable of demonstrating an analgesic effect.

WO00/10598 describes the preparation of modified clostridial neurotoxin molecules that target mucus hypersecreting cells (or neuronal cells controlling said mucus hypersecreting cells), which modified neurotoxins are capable of inhibiting hypersecretion from said cells.

WO01/21213 describes the preparation of modified clostridial neurotoxin molecules that target a wide range of different types of non-neuronal target cells. When so targeted and delivered into the cytosol, the modified molecules are capable of preventing secretion from the target cells.

Additional publications in the technical field of re-targeted toxin molecules include:—WO00/62814; WO00/04926; U.S. Pat. No. 5,773,586; WO93/15766; WO00/61192; WO99/58571; and US2003/0059912.

Thus, from the above-described publications, it will be appreciated that the basic concept of re-targeting a toxin to a desired target cell, by selecting a TM that has a corresponding receptor present on the target cell, has been well documented.

However, not all receptors present on a desired target cell are susceptible to internalisation and subsequent endosome formation. In addition, different receptors present on a target cell of interest demonstrate different binding affinities for different TMs. Thus, a re-targeted toxin conjugate comprising a particular TM may have a low binding affinity for a desired target cell, which is undesirable.

There is therefore a need to develop modified toxin conjugates that address one or more of the above problems.

The present invention relates to the identification of, and use of an “agonist” molecule to re-target a toxin to a cell of therapeutic interest. In particular, the present invention describes a method for designing a toxin conjugate, and describes therapeutic applications of said conjugates to inhibit or reduce cellular processes. Even more particularly, the present invention describes a method for designing toxin conjugates based upon non-cytotoxic toxins able to inhibit exocytosis, such as clostridial neurotoxins, and describes therapeutic applications of said conjugates to inhibit or reduce exocytoels (for example secretion, or the delivery of proteins such as receptors, transporters, and membrane channels to the plasma membrane of a cell).

The process of exocytic fusion involves the movement of cellular vesicles, which move to and fuse with the plasma membrane. Thus, an agent of the present invention is preferably capable of inhibiting delivery and/or fusion of a vesicle from the cytosol of a target cell to the cell membrane of said target cell.

Exocytic fusion may lead to two principal target cell phenotypes, both of which are addressed by the present invention. The first phenotype is secretion, and the second type.

Is membrane protein concentration/density.

Membrane proteins can be conveniently sub-divided into three basic types depending on the function of the membrane protein once delivered to the cell membrane. The three basic types are:—receptors; transporters; and membrane channels. In the context of the present invention, the term “receptor” embraces the related term “acceptor”.

The use of an agonist, which would normally stimulate a biological process, particularly exocytosis (for example, an increase in cellular secretion, or an upregulation in membrane protein expression), is an exciting development in the technical field of re-targeted toxins.

Furthermore, it is particularly surprising that an agonist may be employed in a therapeutic composition to achieve a reduction or inhibition of a biological process that the agonist would normally stimulate.

According to a first aspect, the present invention provides a method of designing (or preparing) a non-cytotoxic, toxin conjugate for inhibition or reduction of exocytic fusion in a target cell, which method comprises:—
(A) identifying an agonist that increases exocytic fusion in said target cell; and
(B) preparing an agent, which agent includes:—
   (i) a Targeting Moiety (TM) that binds the agent to a Binding Site on said target cell, which Binding Site undergoes endocytosis to be incorporated into an endosome within the target cell, and wherein the TM is an agonist identifiable by step (A);
   (ii) a non-cytotoxic protease or a fragment thereof, which protease or protease fragment is capable of cleaving a protein of the exocytic fusion apparatus of said target cell; and
   (iii) a Translocation Domain that translocates the protease or protease fragment from within the endosome, across the endosomal membrane, and into the cytosol of the target cell.

Exocytic fusion is a process by which intracellular molecules are transported from the cytosol of a target cell to the plasma (ie. cell) membrane thereof. Thereafter, the intracellular molecules may become displayed on the outer surface of the plasma membrane, or may be secreted into the extracellular environment.

In a healthy individual, the rate of exocytic fusion is carefully regulated and allows control of the transport of molecules between the cytosol and the plasma membrane of a cell. For example, regulation of the exocytic cycle allows control of the density of receptors, transporters, or membrane channels present at a cell's surface, and/or allows control of the secretion rate of intracellular components (eg. hormones, or neurotransmitters) from the cytosol of the cell.

However, in an unhealthy individual, the regulation of exocytic fusion may be modified. For example, exocytic fusion may cause affected cells to enter a state of hypersecretion.

Alternatively, exocytic fusion may result in the display of an increased concentration of receptors, transporters, or membrane channels present on the cell surface, which may expose the cell in question to undesirable external stimuli. Thus, the process of exocytic fusion may contribute to the progression and/or severity of disease, and therefore provides a target for therapeutic intervention. Examples of such exocytic fusion events include the hypersecretion of mucus, which may contribute to the progression and/or severity of chronic obstructive pulmonary disease (COPD) or asthma; and the upregulation of complement receptors, which may contribute to the progression and/or severity of inflammation.

It should be also appreciated that otherwise normal rates of cellular exocytic fusion may contribute to the progression and severity of disease in compromised patients (eg. immunocompromised patients). Thus, by targeting exocytic fusion in accordance with the present invention, it is also possible to provide therapy in such patients.

The agonist-containing agents of the present invention represent a distinct sub-set of toxin conjugates. In more detail, the agents of the present invention comprise TMs that have been selected on the basis of specific agonist properties rather than on the simple basis that they have a corresponding receptor on a target cell of interest.

The term “agonist” in the context of the present invention embraces any molecule that is capable of increasing exocytic fusion in a target cell.

Preferably, an “agonist” is a peptide or protein molecule that is capable of inducing a target cell into one or more of the following states:—secretion; or an increased concentration of cellular membrane proteins such as receptors or transporters or membrane channels.

Thus, an agonist may be identified by literature review and/or by any method that can directly or indirectly measure cellular secretion, or the concentration/density of a membrane protein (eg. receptors, transporters, or membrane channels) in a target cell. In this regard, the step of “identifying” an agonist preferably includes confirmation that the agonist molecule increases exocytic fusion in the target cell.

In more detail, secretion is readily measurable by detection of an appropriate molecule that has been secreted into the extracellular milieu. This may be performed by a variety of conventional detection methods including:—chromatography; mass spectroscopy; and fluorescence. Preferred methods may include:—ELISA/EIA/RIA techniques; or radio-tracer assays to quantitatively assess the secreted molecules.

Alternatively, any one of a number of conventional assays may be employed to identify a change in concentration or density of a cell membrane protein.

In more detail, for the assessment of a cell membrane receptor concentration, any one of the following techniques may be employed:—Immuno-histochemistry; flow cytometry; quantitative western blotting of isolated plasma membrane cell fractions; and fluorescent-ligand/radio-ligand binding assays. For the assessment of a cell membrane channel concentration, any one of the following techniques may be employed:—biochemical assessment of ion concentration in serum/plasma/urine; electrophysiology of tissue (eg. ex vivo tissue); intra- and extracellular assessment of transported material (eg. glucose); Immuno-histochemistry, flow cytometry; and quantitative western blotting of isolated plasma membrane cell fractions. For the assessment of a cell membrane transporter concentration, any one of the following techniques may be employed:—Immuno-histochemistry; flow cytometry; quantitative western blotting of isolated plasma membrane cell fractions; and intra- and extracellular assessment of transported material (eg. glucose).

Any of the above-mentioned assays are suitable for identifying/confirming that an agonist is capable of increasing exocytic fusion in a target cell, and a number of said assays are illustrated by reference to the Examples of the present application.

In use of the present invention, a target cell is selected in which it is desired to reduce or inhibit the process of exocytic fusion, which exocytic process contributes to the symptoms associated with a medical condition or disease. For example, the target cell in question may demonstrate an undesirable phenotype (eg. an undesirable secretion, or the expression of an undesirable concentration of membrane receptor, transporter or membrane channel), which contributes to the symptoms associated with a medical condition or disease.

Alternatively, a target cell may be selected in which the process of exocytic fusion contributes to the medical condition or disease.

Thus, in addition to the aforementioned assays for confirming that a test molecule is an agonist in the context of the present invention, it is also possible to confirm that a test molecule is an agonist by administering the test molecule in vivo, and then monitoring for an increase in or worsening of the symptoms associated with a condition or disease (or a worsening of the condition/disease itself).

An agonist of the present invention therefore has an effect, which is measurable either on a target cell itself or on the symptoms associated with a medical condition or disease (or on the condition/disease itself).

Conventionally, an agonist has been considered any molecule that can either increase or decrease activities within a cell, namely any molecule that simply causes in an alteration of cell activity. For example, the conventional meaning of an agonist would include:—

a chemical substance capable of combining with a receptor on a cell and initiating a reaction or activity; or

a drug that induces an active response by activating receptors, whether the response is an increase or decrease in cellular activity.

However, for the purposes of this invention, an agonist is more specifically defined as a molecule that is capable of stimulating the process of exocytic fusion in a target cell, which process is susceptible to inhibition by a protease (or fragment thereof) capable of cleaving a protein of the exocytic fusion apparatus in said target cell

Accordingly, the particular agonist definition of the present invention excludes many molecules that may be conventionally considered as agonists. For example, nerve growth factor (NGF) is an agonist in respect of its ability to promote neuronal differentiation via binding to a TrkA receptor. However, NGF is not an agonist when assessed by the above criteria because it is not a principal inducer of exocytic fusion. In addition, the process that NGF stimulates (ie. cell differentiation) is not susceptible to inhibition by the protease activity of a non-cytotoxic toxin molecule.

In use, an agonist-containing agent of the present invention does not deactivate an agonist receptor on a target cell, but rather the protease activity of the agent serves to negate the agonist-mediated response.

Furthermore, once-delivered to the cytosol of a target cell, the protease component of an agent of the present invention inhibits or blocks the action of all subsequent agonists capable of causing the same effect (ie. Increased exocytic fusion) in the same target cell.

This is advantageous and means that the agents of the present invention have application in situations where multiple agonists may be responsible for a given disease or condition.

Thus, when designing an agent of the present invention, the TM that is selected for agent delivery need not necessarily be the principal agonist of the disease/condition that is to be addressed.

In addition to the previously recorded benefits of non-cytotoxic protease-containing therapeutics, such as:—

an extended duration of action (proteases provide potential for significantly extended duration of therapy); a variable duration of action (a particular type of protease may be selected to determine the desired duration of action); and a lack of side-effects (specific targeting to the cell in question leads to decreased side effects compared to conventional small molecule drugs, which are generally less specific);

agonist-mediated delivery according to the present invention provides the following significant advantage over previous non-cytotoxic protease-containing therapeutics:—

use of an agonist may confer preferential binding and/or internalisation properties on the agent. This, in turn, may result in more efficient delivery of the protease component to a target cell.

In addition, use of an agonist as a TM is self-limiting with respect to side-effects. In more detail, binding of an agonist to a target cell increases exocytic fusion, which may exacerbate a medical disease state or a condition. However, the exocytic process that is stimulated by agonist binding is subsequently reduced or inhibited by the protease component of the agent.

As detailed above, the present invention addresses the need for an improved or alternative agent that is capable of inhibiting the process of exocytic fusion in a target cell. As detailed above, this is achieved through use of an agonist as a Targeting Moiety. Thus, the present invention provides use of an agonist that increases exocytic fusion in a target cell, for the manufacture of a medicament for treating the symptoms associated with a medical condition/disease (or the medical condition/disease itself), wherein said symptoms (or the medical condition/disease itself) results from increased exocytic fusion in said target cell.

In use of the present invention, a particularly preferred agonist is a molecule that is capable of stimulating an increase in the cell membrane concentration of one or more of a transporter (such as the GLUT4 transporter in adipose tissue for transport of glucose), a membrane channel (such as the Na⁺ channel in the kidney), a receptor (such as the CD23 IgE receptor on activated monocytes), or stimulating an increase in the secretion of an extracellular mediator (such as mucin following IL13 stimulation of airway goblet cells).

The above-described method for designing an agent of the present invention results in the preparation of a protein-based protease conjugate. As an alternative, said method may be employed to design a DNA-based protease conjugate. Thus, in a corresponding aspect of the present invention there is provided a method of designing a non-cytotoxic toxin conjugate, which method comprises:—
(A) identifying an agonist that increases exocytic fusion in said target cell; and
(B) preparing an agent, which agent includes:—
   (i) a Targeting Moiety (TM) that binds the agent to a Binding Site on said target cell, which Binding Site undergoes endocytosis to be incorporated into an endosome within the target cell, and wherein the TM is an agonist identifiable by step (A);
   (ii) a DNA sequence encoding a non-cytotoxic protease or a fragment thereof, which DNA sequence is expressible in the target cell and when so expressed provides a protease or protease fragment capable of cleaving a protein of the exocytic fusion apparatus of said target cell; and
   (iii) a Translocation Domain that translocates the DNA sequence encoding the protease or protease fragment from within the endosome, across the endosomal membrane, and into the cytosol of the target cell.

The DNA sequence encoding the non-cytotoxic protease component may be expressed under the control of an operably linked promoter present as part of the agent (eg. as part of the protease DNA sequence upstream of the coding region). Alternatively, expression of the protease component in the target cell may rely on a promoter present in the target cell.

The DNA sequence encoding the protease component may integrate into a DNA sequence of the target cell. One or more integration site(s) may be provided as part of the agent (eg. as part of the protease DNA sequence).

The first aspect may further comprise the step of preparing a pharmaceutical composition by combining the agent with a pharmaceutically acceptable carder, diluent and/or excipient.

According to a related embodiment of the first aspect of the present invention there is provided a method of identifying an agonist that is suitable for re-targeting a non-cytotoxic protease or a fragment thereof to a target cell, which protease or protease fragment is to capable of cleaving a protein of the exocytic fusion apparatus of the target cell, said method comprising:—
(A) identifying a putative agonist molecule;
(B) contacting the target cell with said putative agonist molecule; and
(C) confirming that said putative agonist molecule is an agonist by identifying an increase in exocytic fusion in the target cell when said molecule is present compared with when said molecule is absent.

Step (B) is preferably performed in vitro, for example with an isolated sample containing the target cell. Alternatively, step (B) may be performed in vivo.

Suitable assays for confirmation step (C) have been described in detail elsewhere in the present specification.

The above method may further comprise one or more of the following optional steps:—
(D) confirming that the putative agonist molecule or agonist is capable of being combined with a non-cytotoxic protease (or a fragment thereof) and optionally a Translocation Domain to form an agent of the present invention; and/or
(E) confirming that said putative agonist molecule or agonist binds to a Binding Site on the target cell, which Binding Site is susceptible to receptor-mediated endocytosis; and/or
(F) confirming that said putative agonist molecule or agonist is able to deliver a non-cytotoxic protease (or fragment thereof) into the cytosol of a target cell.

The above steps (D)-(F) may be confirmed by routine tests that would be readily available to a skilled person.

For example, step (D) may be performed by a simple chemical conjugation experiment using conventional conjugation reagents and/or linker molecules, followed by native polyacrylamide gel electrophoresis to confirm that an agent of the present invention is formed that has the anticipated molecular weight. The agent components are typically linked together (optionally via linker molecules) by covalent bonds.

For example, step (E) may be performed by any one of a range of methodologies for assessment of binding of a ligand. Standard text, for example “Receptor-Ligand Interactions. A Practical Approach. Ed. E. C. Hulme, IRL Press, 1992” are available that describe such approaches in detail. In brief, the agonist or putative agonist molecule is labelled (for example, with 125-Iodine) and applied to a cell preparation in vitro in the presence of an excess of unlabelled agonist. The purpose of the unlabelled material is to saturate any non-specific binding sites. The agonist is incubated with the cell preparation for sufficient time to achieve equilibrium, and the amount of label bound to the cells assessed by measuring cell associated radioactivity, for example by scintillation or gamma counting.

A further example involves gold-labelling of the agonist (or putative agonist), followed by the use of electron microscopy to monitor the cellular transport progress of the labelled agonist [see the basic methodology described by Rabinowitz S. (1992); J. Cell. Biol. 116(1): pp. 95-112; and that described by van Deurs (1986); J. Cell. Biol. 102: pp. 37-47].

For example, step (F) may be performed by contacting the agent prepared in step (D) with a suitable target cell and assessing cleavage of the substrate. This is performed by extraction of the SNARE proteins, followed by Western blotting of SDS-PAGE-separated samples. Cleavage of substrate is indicative of delivery of the protease into the target cell.

In this regard, cleavage may be monitored by disappearance of substrate and/or appearance of cleavage product. A particularly useful antibody that selectively binds to the cleaved substrate product is described in WO95/33850.

In steps (D) and (F), the Translocation Domain function of the agent may provided by a TM agonist that has dual TM and translocating functions. Conversely, the TM function of the agent may be provided by a Translocation Domain that has dual translocating and TM functions. Alternatively, separate TM and Translocation Domain components may be included.

Targeting Moiety (TM) means any chemical structure associated with an agent that functionally interacts with a Binding Site to cause a physical association between the agent and the surface of a target cell. The term TM embraces any molecule (ie. a naturally occurring molecule, or a chemically/physically modified variant thereof) that is capable of binding to a Binding Site on the target cell, which Binding Site is capable of internalisation (eg. endosome formation)—also referred to as receptor-mediated endocytosis. The TM may possess an endosomal membrane translocation, in which case separate TM and Translocation Domain components need not be present in an agent of the present invention.

An agonist means any molecule that is capable of increasing exocytic fusion in a target cell.

In the context of this invention, the agonist also has TM properties and, as such, functionally interacts with a Binding Site to cause a physical association between the agent and the surface of a target cell.

The term non-cytotoxic means that the protease molecule in question does not kill the target cell to which it has been re-targeted.

The protease of the present invention embraces all naturally-occurring non-cytotoxic proteases that are capable of cleaving one or more proteins of the exocytic fusion apparatus in eukaryotic cells.

The protease of the present invention is preferably a bacterial protease (or fragment thereof). More preferably the bacterial protease is selected from the genera Clostridium or Neisseria (eg. a clostridial L-chain, or a neisserial IgA protease preferably from N. gonorrhoeae).

The present invention also embraces modified non-cytotoxic proteases, which include amino acid sequences that do not occur in nature and/or synthetic amino acid residues, so long as the modified proteases still demonstrate the above-mentioned protease activity.

The protease of the present invention preferably demonstrates a serine or metalloprotease activity (eg. endopeptidase activity). The protease is preferably specific for a SNARE protein (eg. SNAP-25, synaptobrevin/VAMP, or syntaxin).

Particular mention is made to the protease domains of neurotoxins, for example the protease domains of bacterial neurotoxins. Thus, the present invention embraces the use of neurotoxin domains, which occur in nature, as well as recombinantly prepared versions of said naturally-occurring neurotoxins.

Exemplary neurotoxins are produced by clostridia, and the term clostridial neurotoxin embraces neurotoxins produced by C. tetani (TeNT), and by C. botulinum (BoNT) serotypes A-G, as well as the closely related BoNT-like neurotoxins produced by C. beratil and C. butyricum. The above-mentioned abbreviations are used throughout the present specification. For example, the nomenclature BoNT/A denotes the source of neurotoxin as BoNT (serotype A). Corresponding nomenclature applies to other BoNT serotypes.

The term L-chain fragment means a component of the L-chain of a neurotoxin, which fragment demonstrates a metalloprotease activity and is capable of proteolytically cleaving a vesicle and/or plasma membrane associated protein involved in cellular exocytosis.

A Translocation Domain is a molecule that enables translocation of a protease (or fragment thereof) into a target cell such that a functional expression of protease activity occurs within the cytosol of the target cell. Whether any molecule (eg. a protein or peptide) possesses the requisite translocation function of the present invention may be confirmed by any one of a number of conventional assays.

For example, Shone C. (1987) describes an in vitro assay employing liposomes, which are challenged with a test molecule. Presence of the requisite translocation function is confirmed by release from the liposomes of K⁺ and/or labelled NAD, which may be readily monitored [see Shone C. (1987) Eur. J. Biochem; vol. 167(1): pp. 175-180].

A further example is provided by Blaustein R. (1987), which describes a simple in vitro assay employing planar phospholipid bllayer membranes. The membranes are challenged with a test molecule and the requisite translocation function is confirmed by an increase in conductance across said membranes [see Blaustein (1987) FEBS Letts; vol. 226, no. 1: 20 pp. 115-120].

Additional methodology to enable assessment of membrane fusion and thus identification of Translocation Domains suitable for use in the present invention are provided by Methods in Enzymology Vol 220 and 221, Membrane Fusion Techniques, Parts A and B, Academic Press 1993.

The Translocation Domain is preferably capable of formation of ion-permeable pores in lipid membranes under conditions of low pH. Preferably it has been found to use only those portions of the protein molecule capable of pore-formation within the endosomal membrane.

The Translocation Domain may be obtained from a microbial protein source, in particular from a bacterial or viral protein source. Hence, in one embodiment, the Translocation Domain is a translocating domain of an enzyme, such as a bacterial toxin or viral protein.

It is well documented that certain domains of bacterial toxin molecules are capable of forming such pores. It is also known that certain translocation domains of virally expressed membrane fusion proteins are capable of forming such pores. Such domains may be employed in the present invention.

The Translocation Domain may be of a clostridial origin, namely the H_{N} domain (or a functional component thereof). H_{N} means a portion or fragment of the H-chain of a clostridial neurotoxin approximately equivalent to the amino-terminal half of the H-chain, or the domain corresponding to that fragment in the intact H-chain. Examples of suitable clostridial Translocation Domains include:—
Botulinum type A neurotoxin—amino acid residues (449-871)
Botulinum type B neurotoxin—amino acid residues (441-858)
Botulinum type C neurotoxin—amino acid residues (442-866)
Botulinum type D neurotoxin—amino acid residues (446-862)
Botulinum type E neurotoxin—amino acid residues (423-845)
Botulinum type F neurotoxin—amino acid residues (440-864)
Botulinum type G neurotoxin—amino acid residues (442-863)
Tetanus neurotoxin—amino acid residues (458-879)

For further details on the genetic basis of toxin production in Clostridium botulinum and C. tetani, we refer to Henderson et al (1997) in The Clostridia: Molecular Biology and Pathogenesis, Academic press.

The term H_{N} embraces naturally-occurring neurotoxin H_{N} portions, and modified H_{N} portions having amino acid sequences that do not occur in nature and/or synthetic amino acid residues, so long as the modified H_{N} portions still demonstrate the above-mentioned translocation function.

Alternatively, the Translocation Domain may be of a non-clostridial origin (see Table 1). Examples of non-clostridial Translocation Domain origins include, but not be restricted to, the translocation domain of diphtheria toxin [O'Keefe et al., Proc. Natl. Acad. Sci. USA (1992) 89, 6202-6206; Silverman et al., J. Biol. Chem. (1993) 269, 22524-22532; and London, E. (1992) Biochem. Biophys. Acta., 1112, pp. 25-51], the translocation domain of Pseudomonas exotoxin type A [Prior et al. Biochemistry (1992) 31, 3555-3559], the translocation domains of anthrax toxin [Blanke et al. Proc. Natl. Acad. Sci. USA (1996) 93, 8437-8442], a variety of fusogenic or hydrophobic peptides of translocating function [Plank et al. J. Biol. Chem. (1994) 269, 12918-12924; and Wagner et al (1992) PNAS, 89, pp. 7934-7938], and amphiphilic peptides [Murata et al (1992) Biochem., 31, pp. 1986-1992]. The Translocation Domain may mirror the Translocation Domain present in a naturally-occurring protein, or may include amino acid variations so long as the variations do not destroy the translocating ability of the Translocation Domain.

Particular examples of viral Translocation Domains suitable for use in the present invention include certain translocating domains of virally expressed membrane fusion proteins. For example, Wagner et al. (1992) and Murata et al. (1992) describe the translocation (ie. membrane fusion and vesiculation) function of a number of fusogenic and amphiphilic peptides derived from the N-terminal region of influenza virus haemagglutinin. Other virally expressed membrane fusion proteins known to have the desired translocating activity are a translocating domain of a fusogenic peptide of Semliki Forest Virus (SFV), a translocating domain of vesicular stomatitis virus (VSV) glycoprotein G, a translocating domain of SER virus F protein and a translocating domain of Foamy virus envelope glycoprotein. Virally encoded “spike proteins” have particular application in the context of the present invention, for example, the E1 protein of SFV and the G protein of the G protein of VSV.

Use of the Translocation Domains listed in Table 1 includes use of sequence variants thereof. A variant may comprise one or more conservative nucleic acid substitutions and/or nucleic acid deletions or insertions, with the proviso—that the variant possesses the requisite translocating function. A variant may also comprise one or more amino acid substitutions and/or amino acid deletions or insertions, so long as the variant possesses the requisite translocating function.

TABLE 1

| Translocation domain source | Amino acid residues | References |
|---|---|---|
| Diphtheria | 194-380 | Silverman et al., 1994, J. |
| toxin | | Biol. Chem. 269, 22524-22532 |
| | | London E., 1992, Biochem. |
| | | Biophys. Acta., 1113, 25-51 |
| Domain II of | 405-613 | Prior et al., 1992, |
| pseudomonas | | Biochemistry 31, 3555-3559 |
| exotoxin | | Kihara & Pastan, 1994, |
| | | Bioconj Chem. 5, 532-538 |
| Influenza virus | GLFGAIAGFIENGWEGMIDGWYG | Plank et al., 1994, J. Biol. |
| haemagglutinin | and variants thereof | Chem. 269, 12918-12924 |
| | | Wagner et al., 1992, PNAS, 89, 7934-7938 |
| | | Murata et al., 1992, |
| | | Biochemistry 31, 1986-1992 |
| Semliki Forest virus | Translocation domain | Kielian et al., 1996, J Cell |
| fusogenic protein | | Biol. 134(4), 863-872 |
| Vesicular Stomatitis | 118-139 | Yao et al., 2003, Virology |
| virus glycoprotein G | | 310(2), 319-332 |
| SER virus F | Translocation domain | Seth et al., 2003, J Virol |
| protein | | 77(11) 6520-6527 |
| Foamy virus envelope | Translocation domain | Picard-Maureau et al., |
| glycoprotein | | 2003, J Virol. 77(8), 4722-4730 |

According to a second aspect of the present invention there is provided a composition, which includes:—
(A) an agent comprising:—
   (i) a Targeting Moiety (TM) that binds the agent to a Binding Site on a target cell, which Binding Site undergoes endocytosis to be incorporated into an endosome within the target cell, and wherein the TM is an agonist that is capable of increasing exocytic fusion in the target cell;
   (ii) a non-cytotoxic protease or a fragment thereof, which protease or protease fragment is capable of cleaving a protein of the exocytic fusion apparatus of said target cell; and
   (iii) a Translocation Domain that translocates the protease or protease fragment from within the endosome, across the endosomal membrane, and into the cytosol of the target cell.

The above-defined components of the agent may be selected and tested in accordance with the details provided for the first aspect of the present invention.

The composition may further comprise:—
(B) an inhibitor that alleviates, in a patient, clinical symptoms caused by increased exocytic fusion.

In a particularly preferred embodiment, the inhibitor alleviates, in a patient, clinical symptoms caused by increased exocytic fusion resulting from binding of the agonist to the target cell.

The term alleviating is used interchangeably with reducing, ameliorating or inhibiting. Thus, the inhibitor may be capable of reducing or ameliorating the symptoms that are induced by agonist binding to a target cell.

The inhibitor component is principally concerned with minimising any undesirable symptoms caused by binding of the agonist, more specifically the TM component of an agent, to a target cell in this regard, the agonist component of an agent in use, causes an initial increase in the rate of exocytic fusion in a target cell. This agonist-induced exocytic fusion may cause short-term undesirable symptoms, and it is these undesirable symptoms with which the inhibitor component is primarily concerned.

The phrase “symptoms caused by (resulting from) increased exocytic fusion” embraces clinical symptoms that are the direct result of agonist binding to a target cell, and clinical symptoms that result from a cascade of cellular events initiated by agonist binding to a target cell.

Accordingly, a composition of the present invention provides a new and desirable means for delivering a non-cytotoxic protease activity into a cell of interest by use of a molecule (ie. the TM agonist), which may provide a stimulation, though short-term, of the cellular process (ie. exocytic fusion) that has been selected as the target for inhibition.

In a preferred embodiment, the composition is for treatment of a medical condition or disease in a patient, preferably in a human. In this embodiment, the inhibitor (when present) is a molecule that alleviates the symptoms associated with said medical condition or disease, preferably the symptoms that have been caused or stimulated by binding of an agent of the present invention to a target cell. In this regard, binding of an agent to a target cell may cause a temporary stimulation of exocytic fusion in said target cell.

The inhibitor may be any conventional pharmaceutical molecule, so long as it is capable of alleviating the symptoms associated with the medical condition/disease that is to be treated. Preferably the inhibitor is capable of alleviating symptoms, which are typically short term symptoms, resulting from increased exocytic fusion in a target cell caused by binding of the agonist TM to said target cell.

Inhibitors may be identified by consultation of the relevant pharmacological and medical texts, and by consultation with medical practitioners. For example, the British National Formulary (published by the British Medical Society and The Royal Pharmaceutical Society of Great Britain) provides listings of approved pharmaceutical products that would be suitable for use in the invention.

The inhibitor preferably has a short-acting duration of action once administered to a patient, for example 1-3 days, preferably 1-2 days, more preferably 24-36 hours. After this period, the non-cytotoxic protease effectiveness provided by the agent increases and the inhibitor effect is no longer necessary; In contrast to the preferred short-acting duration of the inhibitor effect, the effect of the agent (ie. the non-cytotoxic protease activity) is typically longer lasting. For example, 1-6 months, preferably 2-4 months.

The inhibitor is advantageously required for a short period following initiation of therapy with an agent to alleviate any short term symptoms caused by binding of the agonist TM.

Subsequently, as a result of inhibition of exocytic fusion by protease action, the effect of the agonist TM is blocked and an inhibitor is no longer required. The effects of the protease are however long lasting and alleviate the disease or condition to be treated for a considerable period of time (weeks, or months), without requiring further use of inhibitor or agent. Thus, the agent of the present invention provides an improved therapy for diseases and reduces the requirement for therapeutic intervention.

In one embodiment, the inhibitor causes an inhibition or reduction of the process of exocytic fusion in the target cell, and provides a short term block of exocytosis. Such an inhibitor preferably does not bind to the Binding Site to which the agent of the invention binds. Thus, there is no substantial competition between an agent of the present invention and the inhibitor component for the Binding Site. The inhibitor should therefore not function as an antagonist of the TM binding activity.

In another embodiment, the inhibitor acts on one or more components secreted from an agonist-stimulated target cell, thereby minimising down-stream effects that would be otherwise induced by the secreted components. For example, the inhibitor may bind to and inactivate a secreted component. Thus, the inhibitor may act at a site away from the target cell to which the agent binds. Alternatively, the inhibitor may be an antagonist of the secreted component(s), thereby blocking the biological activity of the secreted components.

In a further embodiment, the inhibitor acts directly on a stimulated target cell to antagonise the stimulated phenotype. For example, when the stimulated phenotype is an increased concentration of a cell membrane protein (eg. a receptor, or a transport channel), the inhibitor may block the receptor or channel in question, thereby reducing or minimising the functional or phenotypic consequence of said receptor or channel being expressed at the cell surface.

In yet another embodiment the inhibitor acts to prevent the signal transduction mechanism of the Binding site for the agonist TM, without affecting the binding of the agonist TM or its internalisation. In this manner, the inhibitor prevents an unwanted short term phenotypic response in the target cell without preventing binding of the agonist TM.

In yet another further embodiment, the inhibitor may function through secondary antagonism, namely binding to a target cell distinct and separate from the target cell of the agent, which causes the release of, or potentiation of a second molecule. The second molecule then acts as an inhibitor through the mechanisms described above for inhibitors acting directly to counter the effects of the agonist TM.

The second aspect is now described with reference to medical conditions or diseases that are addressed by the present invention.

In use, the compositions of the present invention are suited for the treatment of diseases that result from undesirable exocytic activity (for example secretion, or the delivery of proteins such as receptors, transporters, and membrane channels to the plasma membrane of a cell) in cells such as, but not limited to endocrine cells, exocrine cells, inflammatory cells, cells of the immune system, cells of the cardiovascular system, bone cells and neuronal cells.

For example, the compositions of the present invention have utility for the treatment of chronic obstructive pulmonary disorder through prevention of secretion of mucus from mucus releasing cells; for the treatment of obesity through prevention of presentation of the glucose transporter GLUT4 in the plasma membrane of adipose cells; for the treatment of allergy through prevention of secretion of mediators from mast cells; or for the treatment of chronic inflammatory conditions through prevention of release of selectins from endothelial cells.

Preparation of an agent according to the present invention is now briefly discussed.

In use of the invention, a Targeting Moiety (TM) provides specificity for the BS on the relevant target cell/s. The TM component of the agent may comprise one of many cell-binding molecules so long as said TM is an agonist as hereinbefore defined. Thus, the TM may include, but is not limited to, lectins, hormones, cytokines, growth factors, peptides, carbohydrates, lipids, glycans, nucleic acids, interleukins (eg. IL-4 and IL-13), TNF (eg. TNF-α), insulin, MCD, and complement components.

It is known in the art that the H_{C} portion of a neurotoxin molecule can be removed from the other portion of the H-chain, known as H_{N}, such that the H_{N} fragment remains disulphide linked to the L-chain of the neurotoxin providing a fragment known as LH_{N}. Thus, in one embodiment of the present invention the LH_{N} fragment of a neurotoxin is covalently linked, using linkages which may include one or more spacer regions, to a TM.

In another embodiment of the invention, the H_{c} domain of a neurotoxin is mutated, blocked or modified, eg. by chemical modification, to reduce or preferably incapacitate its ability to bind the neurotoxin to receptors at the neuromuscular junction. This modified neurotoxin is then covalently linked, using linkages which may include one or more spacer regions, to a TM.

In another embodiment of the invention, the H-chain of a neurotoxin, in which the H_{C} domain is mutated, blocked or modified, eg. by chemical modification, to reduce or preferably incapacitate its native binding ability, is combined with the L-chain of a different neurotoxin, or another protease capable of cleaving a protein of the exocytic fusion apparatus (eg. IgA protease of N. gonorrhoeae). This hybrid, modified neurotoxin is then covalently linked, using linkages which may include one or more spacer regions, to a TM.

In another embodiment of the invention, the H_{N} domain of a neurotoxin is combined with the L-chain of a different neurotoxin, or another protease capable of cleaving a protein of the exocytic fusion apparatus (eg. IgA protease of N. gonorrhoeae). This hybrid is then covalently linked, using linkages which may include one or more spacer regions, to a TM.

In another embodiment of the invention, the protease (for example the L-chain component of a neurotoxin) is covalently linked, using linkages that may include one or more spacer regions, to a TM that can also effect the internalisation of the protease into the cytoplasm of the relevant target cell/s.

In another embodiment of the invention, the protease (for example the L-chain component of a neurotoxin) is covalently linked, using linkages which may include one or more spacer regions, to a translocation domain to effect transport of the protease fragment into the cytosol.

In use, the domains of an agent according to the present invention are associated with each other. In one embodiment, two or more of the Domains may be joined together either directly (eg. by a covalent linkage), or via a linker molecule.

Conjugation techniques suitable for use in the present invention have been well documented, and include:—Chemistry of protein conjugation and cross-linking. Edited by Wong, S. S. 1993, CRC Press Inc., Florida; and Bioconjugate techniques, Edited by Hermanson, G. T. 1996, Academic Press, London, UK.

The agents according to the present invention may be prepared recombinantly.

In one embodiment, the preparation of a recombinant agent involves arrangement of the coding sequences of the selected TM and protease component in a single genetic construct. These coding sequences may be arranged in-frame so that subsequent transcription and translation is continuous through both coding sequences and results in a fusion protein. All constructs would have a 5′ ATG codon to encode an N-terminal methionine, and a C-terminal translational stop codon.

Thus, a L-chain of a clostridial neurotoxin or another protease capable of cleaving a protein of the exocytic fusion apparatus (eg an IgA protease), or a fragment/variant thereof, may be expressed recombinantly as a fusion protein with a TM, which TM can also effect the internalisation of the L-chain component into the cytoplasm of the relevant target cell/s responsible for secretion. Alternatively, the fusion protein may further comprise a Translocation Domain. The expressed fusion protein may include one or more spacer regions.

By way of example, the following information is required to produce, recombinantly, an agent of the present invention:—
(I) DNA sequence data relating to a selected TM;
(II) DNA sequence data relating to the protease component;
(III) DNA sequence data relating to the translocation domain; and
(IV) a protocol to permit construction and expression of the construct comprising (I), (II) and (ill).

All of the above basic information (I)-(IV) are either readily available, or are readily determinable by conventional methods. For example, both WO98/07864 and WO99/17806 exemplify recombinant technology suitable for use in the present application.

In addition, methods for the construction and expression of the constructs of the present invention may employ information from the following references and others:—
Lorberboum-Galski, H., FitzGerald, D., Chaudhary, V., Adhya, S., Pastan, I. (1988). Cytotoxic activity of an interleukin 2-Pseudomonas exotoxin chimeric protein produced in Escherichia coli. Proc Natl Acad Sci USA 85(6):1922-6;
Murphy, J. R. (1988) Diphtheria-related peptide hormone gene fusions: a molecular genetic approach to chimeric toxin development. Cancer Treat Res; 37:123-40;
Williams, D. P., Parker, K., Bacha, P., Bishai, W., Borowski, M., Genbauffe, F., Strom, T. B., Murphy, J. R. (1987). Diphtheria toxin receptor binding domain substitution with interleukin-2: genetic construction and properties of a diphtheria toxin-related interleukin-2 fusion protein. Protein Eng; 1(6):493-8;
Arora, N., Williamson, L. C., Leppla, S. H., Halpern, J. L. (1994). Cytotoxic effects of a chimeric protein consisting of tetanus toxin light chain and anthrax toxin lethal factor in non-neuronal cells J Biol Chem, 269(42):26165-71;
Brinkmann, U., Reiter, Y., Jung, S. H., Lee, B., Pastan, I. (1993). A recombinant immunotoxin containing a disulphide-stabilized Fv fragment. Proc Nati Acad Sci USA; 90(16):7538-42; and
O'Hare, M., Brown, A. N., Hussain, K., Gebhardt, A., Watson, U., Roberts, L. M., Vitetta, E. S., Thorpe, P. E., Lord, J. M. (1990). Cytotoxicity of a recombinant ricin-A-chain fusion protein containing a proteolytically-cleavable spacer sequence. FEBS Lett October 29; 273(1-2):200-4.
Suitable clostridial neurotoxin sequence information relating to L- and LH_{N}-chains may be obtained from, for example, Kurazono, H. (1992) J. Biol. Chem., vol. 267, No. 21, pp. 14721-14729; and Popoff, M. R., and Marvaud, J.-C. (1999) The Comprehensive Sourcebook of Bacterial Protein Toxins, 2nd edition (ed. Alouf, J. E., and Freer, J. H.), Academic Press, pp. 174-201.

All of the aforementioned publications are hereby incorporated into the present specification by reference thereto.

Similarly, suitable TM sequence data are widely available in the art. Alternatively, any necessary sequence data may be obtained by techniques which are well-known to the skilled person.

For example, DNA encoding the TM component may be cloned from a source organism by screening a cDNA library for the correct coding region (for example by using specific oligonucleotides based on the known sequence information to probe the library), isolating the TM DNA, sequencing this DNA for confirmation purposes, and then placing the isolated DNA in an appropriate expression vector for expression in the chosen host.

As an alternative to isolation of the sequence from a library, the available sequence information may be employed to prepare specific primers for use in PCR, whereby the coding sequence is then amplified directly from the source material and, by suitable use of primers, may be cloned directly into an expression vector.

Another alternative method for isolation of the coding sequence is to use the existing sequence information and synthesise a copy, possibly incorporating alterations, using DNA synthesis technology. For example, DNA sequence data may be generated from existing protein and/or RNA sequence information. Using DNA synthesis technology to do this (and the alternative described above) enables the codon bias of the coding sequence to be modified to be optimal for the chosen expression host. This may give rise to superior expression levels of the fusion protein.

Optimisation of the codon bias for the expression host may be applied to the DNA sequences encoding the TM and clostridial components of the construct. Optimisation of the codon bias is possible by application of the protein sequence into freely available DNA/protein database software, eg. programs available from Genetics Computer Group, Inc.

The agent or agent plus inhibitor compositions of the present invention are suitable for use in treating various medical conditions or diseases, as described above (see, in particular, the first and second aspect of the present invention). Thus, the compositions may include a pharmaceutically acceptable carrier.

In use, the agent of the present invention may be administered prior to, simultaneously with, or subsequent to the inhibitor.

In use, the agent and/or inhibitor are typically employed in the form of a pharmaceutical composition in association with a pharmaceutical carrier, diluent and/or excipient, although the exact form of the composition may be tailored to the mode of administration.

Administration is preferably to a mammal, more preferably to a human.

The components (ie. agent, and/or inhibitor) may, for example, be employed in the form of an aerosol or nebulisable solution for inhalation or a sterile solution for parenteral administration, intra-articular administration or intra-cranial administration.

For treating endocrine targets, i.v. injection, direct injection into gland, or aerosolisation for lung delivery are preferred; for treating inflammatory cell targets, i.v. injection, sub-cutaneous injection, or surface patch administration or aerosolisation for lung delivery are preferred; for treating exocrine targets, i.v. injection, or direct injection into or direct administration to the gland or aerosolisation for lung delivery are preferred; for treating immunological targets, i.v. injection, or injection into specific tissues eg. thymus, bone marrow, or lymph tissue are preferred; for treatment of cardiovascular targets, i.v. injection is preferred; and for treatment of bone targets, i.v. injection, or direct injection is preferred. In cases of i.v. injection, this should also include the use of pump systems. In the case of compositions for treating neuronal targets, spinal injection (eg. epidural or intrathecal) or indwelling pumps may be used.

The dosage ranges for administration of the components of the present invention are those to produce the desired therapeutic effect. It will be appreciated that the dosage range required depends on the precise nature of the components, the route of administration, the nature of the formulation, the age of the patient, the nature, extent or severity of the patient's condition, contraindications, if any, and the judgement of the attending physician.

Suitable daily dosages (for each component) are in the range 0.0001-1 mg/kg, preferably 0.0001-0.5 mg/kg, more preferably 0.002-0.5 mg/kg, and particularly preferably 0.004-0.5 mg/kg. The unit dosage can vary from less that 1 microgram to 30 mg, but typically will be in the region of 0.01 to 1 mg per dose, which may be administered daily or preferably less frequently, such as weekly or six monthly.

Wide variations in the required dosage, however, are to be expected depending on the precise nature of the components, and the differing efficiencies of various routes of administration. For example, oral administration would be expected to require higher dosages than administration by intravenous injection.

Variations in these dosage levels can be adjusted using standard empirical routines for optimisation, as is well understood in the art.

Compositions suitable for injection may be in the form of solutions, suspensions or emulsions, or dry powders which are dissolved or suspended in a suitable vehicle prior to use.

Fluid unit dosage forms are typically prepared utilising a pyrogen-free sterile vehicle. The active ingredients, depending on the vehicle and concentration used, can be either dissolved or suspended in the vehicle.

Solutions may be used for all forms of parenteral administration, and are particularly used for intravenous injection. In preparing solutions the components can be dissolved in the vehicle, the solution being made isotonic if necessary by addition of sodium chloride and sterilised by filtration through a sterile filter using aseptic techniques before filling into suitable sterile vials or ampoules and sealing. Alternatively, if solution stability is adequate, the solution in its sealed containers may be sterilised by autoclaving.

Advantageously additives such as buffering, solubilising, stabilising, preservative or bactericidal, suspending or emulsifying agents and/or local anaesthetic agents may be dissolved in the vehicle.

Dry powders which are dissolved or suspended in a suitable vehicle prior to use may be prepared by filling pre-sterilised drug substance and other ingredients into a sterile container using aseptic technique in a sterile area.

Alternatively the components (ie. agent plus inhibitor) and other ingredients may be dissolved in an aqueous vehicle, the solution is sterilized by filtration and distributed into suitable containers using aseptic technique in a sterile area. The product is then freeze dried and the containers are sealed aseptically.

Parenteral suspensions, suitable for intramuscular, subcutaneous or intradermal injection, are prepared in substantially the same manner, except that the sterile components are suspended in the sterile vehicle, instead of being dissolved and sterilisation cannot be accomplished by filtration. The components may be isolated in a sterile state or alternatively it may be sterilised after isolation, eg. by gamma irradiation.

Advantageously, a suspending agent for example polyvinylpyrrolidone is included in the composition/s to facilitate uniform distribution of the components.

Compositions suitable for administration via the respiratory tract include aerosols, nebulisable solutions or microfine powders for insufflation. In the latter case, particle size of less than 50 microns, especially less than 10 microns, is preferred. Such compositions may be made up in a conventional manner and employed in conjunction with conventional administration devices.

The compositions (ie. agent with or without inhibitor) described in this invention can be used in vivo, either directly or as a pharmaceutically acceptable salt, for the treatment of conditions involving exocytosis (for example secretion, or the delivery of proteins such as receptors, transporters, and membrane channels to the plasma membrane of a cell).

The present invention is now described by reference to the following Examples and Figures, without intended limitation thereto.
Example 1 Assessment of IL13 agonist activity
Example 2 Expression & purification of catalytically active recLH_{N}/C
Example 3 Production of a conjugate of IL13 and LH_{N}/C
Example 4 Production of single polypeptide fusion conjugate of IL13 and LH_{N}/C
Example 5 Activity of IL13-LH_{N}/C conjugate in mucus releasing cells
Example 6 Activity of IL13-LH_{N}/C in an ex vivo model of COPD
Example 7 In vivo efficacy of IL13-LH_{N}/C in reducing the symptoms of COPD
Example 8 Production of single polypeptide fusion of IL13-IgA protease
Example 9 Assessment of agonist activity of insulin
Example 10 Expression & purification of catalytically active recLH_{N}/B
Example 11 Production of an insulin-LH_{N}/B conjugate
Example 12 Activity of insulin-LH_{N}/B in adipose cells
Example 13 In vivo efficacy of insulin-LH_{N}/B in reducing the symptoms of obesity
Example 14 Assessment of agonist activity of mast cell degranulating peptide (MCD peptide)
Example 15 Production of single polypeptide fusion of MCD peptide and LH_{N}/C
Example 16 Activity of MCD peptide-LH_{N}/C mast cells
Example 17 In vivo efficacy of MCD peptide -LH_{N}/C in reducing the symptoms of asthma
Example 18 Assessment of IL4 agonist activity
Example 19 Production of single polypeptide fusion of IL4-LH_{N}/C
Example 20 Activity of IL4-LH_{N}/C in preventing surface expression of the IgE receptor
CD23 in human monocytes
Example 21 Assessment of TNFα agonist activity
Example 22 In vivo efficacy of TNFα-LH_{N}/C in reducing the symptoms of inflammation
Example 23 Assessment of agonist activity of insulin increasing presentation of NMDA channels in hippocampal and cerebral cortex neurons
Example 24 Production of a conjugate for delivery of DNA encoding LC/C into a cell

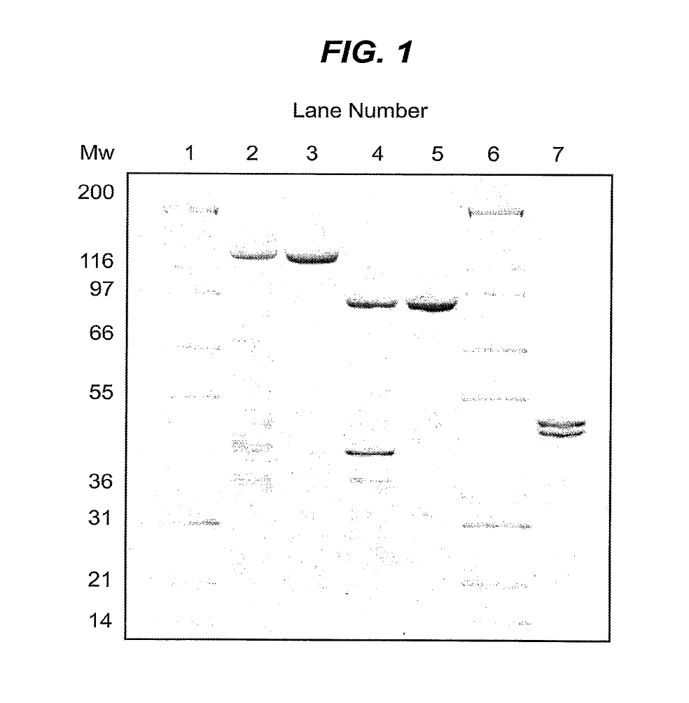

FIG. 1 shows SDS-PAGE analysis of expression and purification of LH_{N}/C from E. coli

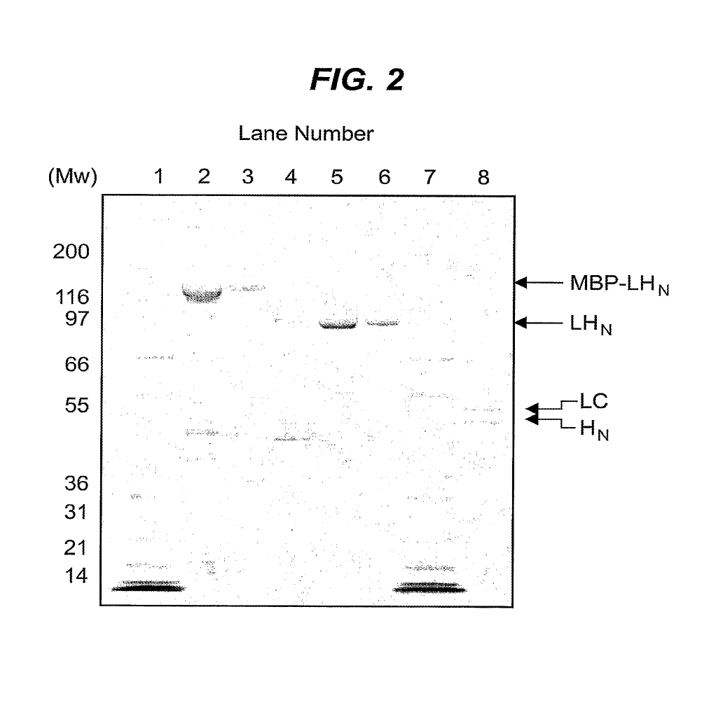

FIG. 2 shows SDS-PAGE analysis of expression and purification of recLH_{N}/B from E. coli

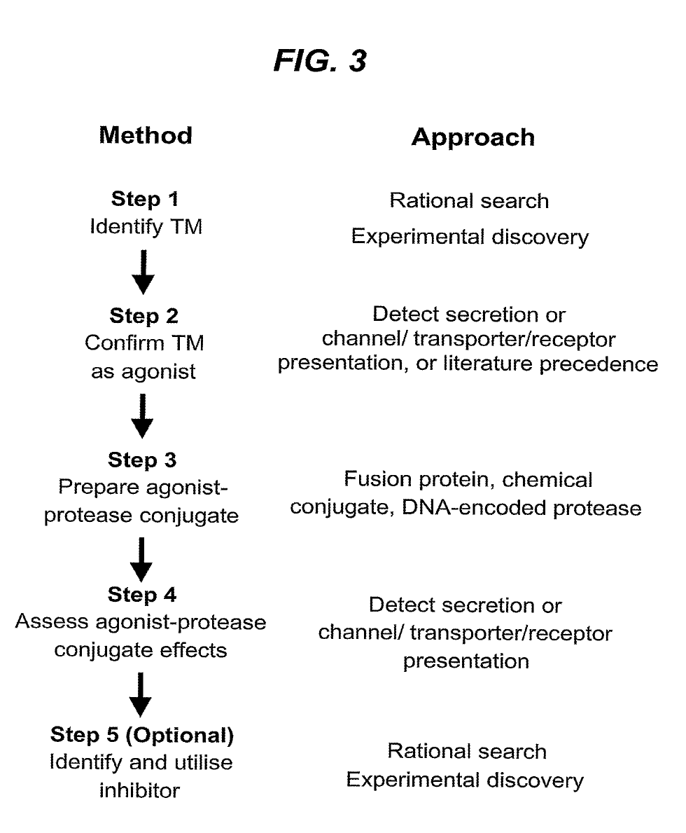

FIG. 3 shows, in a 5-step flow diagram form, a preferred method of the present invention:—
Step 1 Identify TM (eg. from rational search such as literature review, from experimental discovery, or by unexpected observation);
Step 2 Confirm that the TM of Step 1 is an agonist by appropriate assay to and/or literature confirmation;
Step 3 Prepare an agent of the present invention by conjugating the agonist (confirmed by Step 2) to a protease component (eg. by chemical or recombinant fusion);
Step 4 Assess the effects of the agonist-containing agent (prepared by Step 3) on secretion and/or membrane protein presentation; and
Step 5 Where, in Step 4, the binding of agent to a target cell causes a short-term increase in symptoms associated with increased exocytic fusion, use is made of all available sources of information (eg. medical texts, current best medical practice) to identify and utilise (an) inhibitor(s) to minimise said short-term side effect(s).

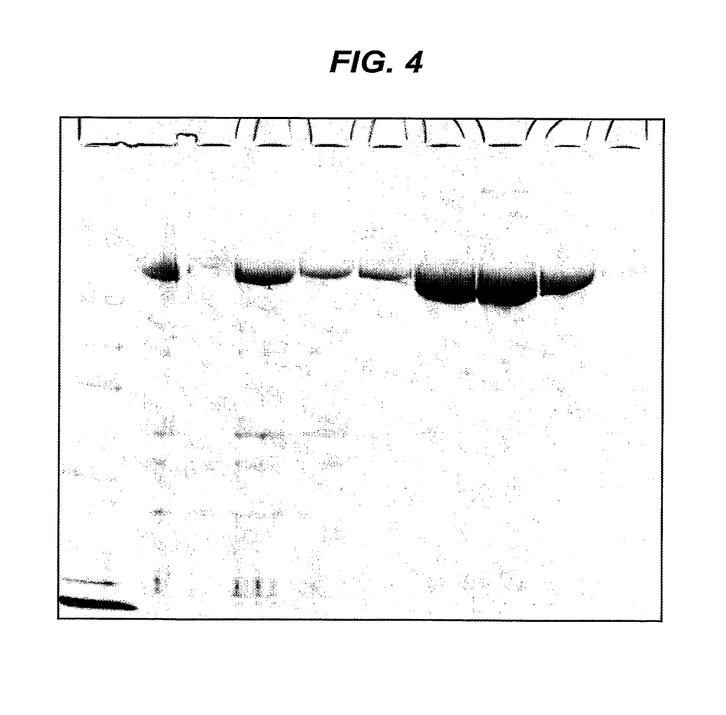

FIG. 4 illustrates the initial capture of MBP-tagged LH_{N}/C-EGF. The order of lanes 1-10 is: Mark 12 marker (Invitrogen); homogenate; pellet (insoluble); load (soluble); amylose column flowthrough; maltose-elution fractions A5, A6, A7, A9, A12.

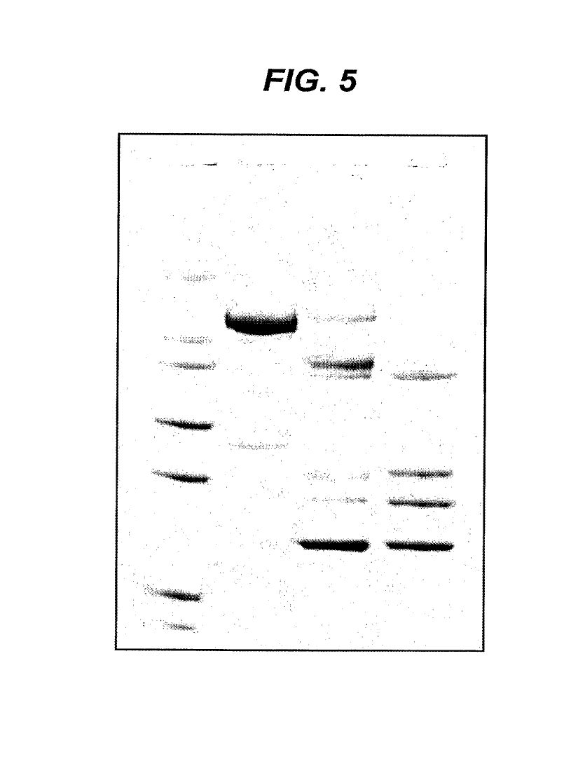

FIG. 5 shows an SDS-PAGE gel illustrating the treatment of fusion protein with Factor Xa to activate the LH_{N}/C. Lanes are identified from left to right as: Mark 12 molecular markers (Invitrogen); LH_{N}/C-EGF fusion in the absence of Factor Xa; LH_{N}/C-EGF fusion after Factor Xa treatment; LH_{N}/C-EGF fusion after Factor Xa treatment in the presence of DTT.

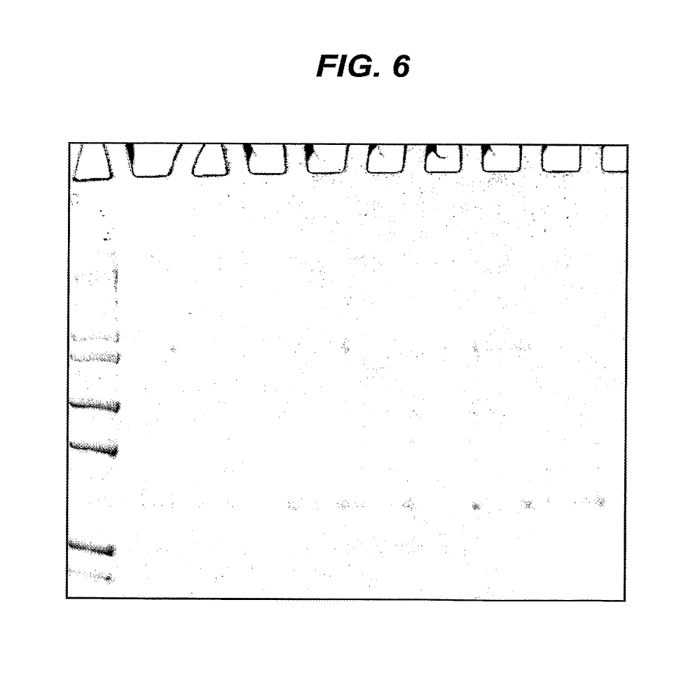

FIG. 6 shows an SDS-PAGE gel illustrating final the LH_{N}/C-EGF fusion product in the absence and presence of DTT. From left to right, the lanes are identified as: Mark 12 molecular markers (Invitrogen); 5 μl fusion; 5 μl fusion plus DTT; 10 μl fusion; 10 μl fusion plus DTT; 20 μl fusion; 20 μl fusion plus DTT.

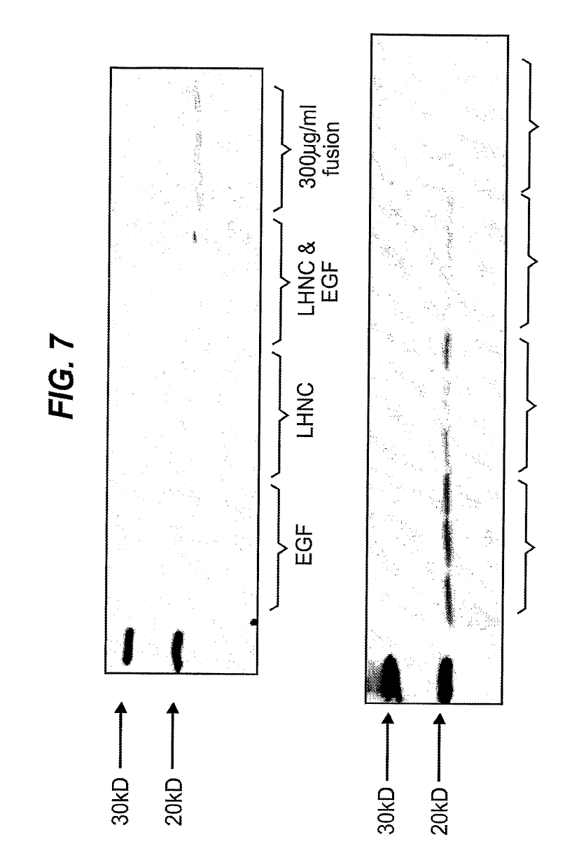

FIG. 7 illustrates the SDS-PAGE and Western Blot analysis described in Example 26.

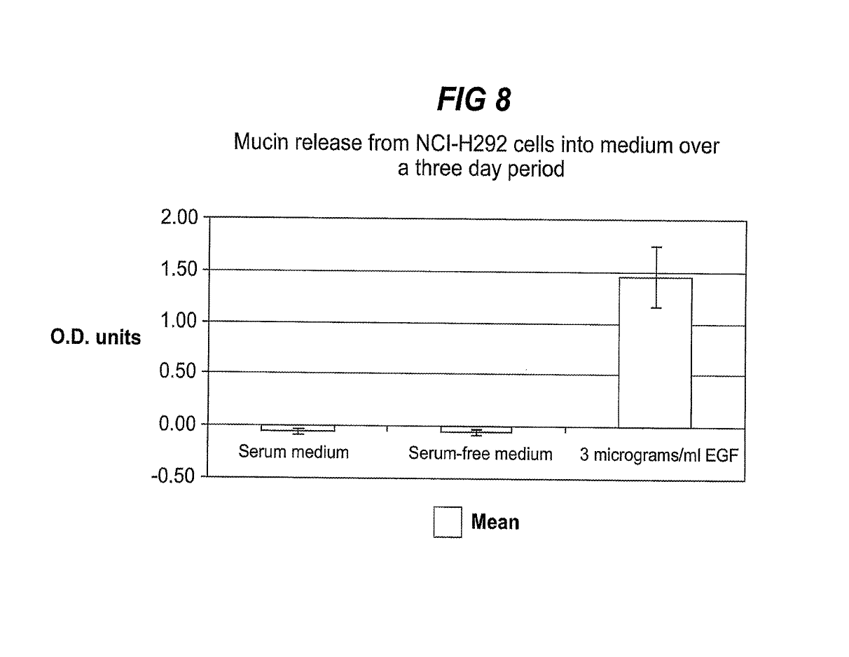

FIG. 8 illustrates mucin release from NCI-H292 cells into medium over a three day period following challenge of said cells with EGF as described in Example 27.

FIGS. 1-2 are now described in more detail.

Referring to FIG. 1, recLH_{N}/C is purified from E. coli cell paste using a two-step strategy described in Example 2. Protein samples are separated by SDS-PAGE and visualised by staining with coomassie blue. Clarified Crude cell lysate (lane 2) is loaded onto Q-Sepharose FF anion-exchange resin. Fusion protein, MBP-LH_{N}/C is eluted with 0.1M NaCl (lane 3). Eluted material incubated at 22° C. for 16 h with factor Xa protease (New England Biolabs) to cleave fusion tag MBP and nick recLH_{N}IC at the linker site. The protein of interest is further purified from cleaved fusion products (lane 4) using Q-Sepharose FF. Lanes 5 and 7 show purified recLH_{N}/C under non-reducing conditions and reduced with 10 mM DTT respectively, to illustrate disulphide bonding at the linker region between LC and H_{N} domains after nicking with factor Xa. Lanes 1 and 6 represent molecular mass markers (shown in KDa); Mark 12 (Invitrogen).

Referring to FIG. 2, recLH_{N}/B is purified from cell paste using a three column strategy as described in Example 10. Protein samples are separated by SDS-PAGE and visualised by staining with simplyblue safestain coomassie reagent. Crude, soluble MBP-LH_{N}/B fusion protein contained within the clarified extract (lane 2) is loaded onto Q-Sepharose FF anion-exchange resin. Lane 3 represents recombinant MBP-LH_{N}/B fusion eluted from column at 150-200 mM salt. This sample is treated with factor Xa protease to remove MBP affinity tag (lane 4), and cleaved mixture diluted to lower salt concentration prior to loading onto a Q-Sepharose FF anion-exchange column. Material eluted between 120-170 mM salt was rich in LH_{N}/B (lane 5). Protein in lane 6 and 8 represents LH_{N}/B harvested after treatment with enterokinase and final purification using Benzamidine Sepharose, under non-reducing and reducing conditions respectively. Lanes 1 and 7 represent molecular mass markers (Mark 12 [invitrogen]).

### EXAMPLE 1—ASSESSMENT OF IL13 AGONIST ACTIVITY

In order to confirm that IL13 is an agonist, i.e. that IL13 increases exocytic fusion in a target cell, the effect of IL13 on release of mucins from in vitro cultures of the human colonic epithelial cell line LS180, and the normal human tracheo-bronchial epithelial (NHTBE) cell line is measured. When IL13 is applied to LS180 and NHTBE cells, there is a marked increase in release of mucin, as measured by an ELISA specific for MUC5AC

Materials

Human IL-13 is obtained from Sigma.

Anti-MUC5AC antisera are obtained from Neomarkers (clone 1-13M1).

LS180 cells are obtained from European Collection of Animal Cell Cultures.

NHTBE cells are obtained from Clonetics.

Methods

LS180 cells are seeded onto 24 well plates and cultured in MEM-Glutamax medium (Gibco) containing 10% foetal bovine serum, 2 mM L-glutamine, 1% pen-Strep, 1% NEAA, 1% HEPES, 1% sodium bicarbonate for 3 days prior to use. IL13 is applied to the cells, and the release of MUC5AC mucin assayed 24 hours later by ELISA.

NHTBE cells are cultured as described by Gray et al. Am. J. Respir. Cell Biol., 14, 104-112 (1996). Briefly, P2 cells are seeded into Transwell-COL collagen coated membrane supports (12 well) and cultured in bronchial epithelial cell growth medium (BEGM) for 7 days. On day 8 the media above the membrane is removed to create an air-liquid interface and the cells are cultured for a further 4 weeks, by when cillia have developed. The cultures are then ready for experimental use. IL13 is applied to the cells, and the release of MUC5AC mucin assayed 24 hours later by ELISA.

For the ELISA the superfusates are removed from the cells to eppendorfs on ice. The cells are then lysed with 450 μl of 0.2M NaOH/well, for 10 minutes at room temp. and neutralised with 450 μl 0.2M HCL and 100 μl HEPES. The cells are scraped from the plate, and the lysate removed to eppendorfs on ice. All samples are stored at −20° C. until assay.

The samples are thawed at 4° C., centrifuged at 13,000×g for 10 min. and the ELISA performed. One hundred μl of supernatant is pipetted, in duplicate, from each tube to a 96 well maxisorp plate (Nunc). Fifty μl of assay buffer is used as a blank. The plate is placed in a 40° C. oven overnight, or until dry and then washed three times in PBS and blotted dry.

The plate is blocked with 100 μl PBS containing 2% BSA, fraction V for 1 hour on a shaker at room temperature and then, again, washed three times in PBS and blotted dry. The plate is then incubated with 50 μl of anti MUC5AC (clone 1-13M1, Neomarkers) 1:1000, diluted in PBST (0.05% tween) for 1 hour on a shaker at room temperature, washed three times in PBS and blotted dry. One hundred μl of horseradish peroxidase anti-mouse IgG (1:2000) is added to each well, incubated for 1 hour on a shaker at room temperature, the plate washed three times in PBS and blotted dry. Two hundred μl of TMB is added to each well, colour allowed to develop, and then 50 μl of 0.5M HCl added to stop the reaction. The final colour reaction is read at 450 nm.

### EXAMPLE 2—EXPRESSION AND PURIFICATION OF CATALYTICALLY ACTIVE RECOMBINANT LH_{N}IC

The coding region for LH_{N}/C is inserted in-frame to the 3′ of the gene encoding maltose binding protein (MBP) in the expression vector pMAL (New England Biolabs) to create pMAL-c2x-LH_{N}/C. In this construct the expressed MBP and LH_{N}/C polypeptides are separated by a Factor Xa cleavage site.

pMAL-c2x-LH_{N}/C is transformed into E. coli AD494 (DE3, IRL) and cultured in Terrific broth complex medium in 8 L fermentor systems. Pre-induction bacterial growth are maintained at 30° C. to an OD600 nm of 8.0, at which stage expression of recMBP-c2x-LH_{N}/C is induced by addition of IPTG to 0.5 mM and a reduction in temperature of culture to 25° C. After 4 hr at 25° C. the bacteria are harvested by centrifugation and the resulting paste stored at −70° C.

The cell paste is resuspended in 50 mM Hepes pH 7.2, 1 μM ZnCl₂ at 1:6 (w/v) and cell disruption is achieved using an APV-Gaulin lab model 1000 homogeniser or a MSE Soniprep 150 sonicator. The resulting suspension is clarified by centrifugation prior to purification.

Following cell disruption and clarification, the MBP-fusion protein is separated on a Q-Sepharose Fast Flow anion-exchange resin in 50 mM Hepes pH 7.2, 1 ìM ZnCl₂ and eluted with the same buffer plus 100 mM NaCl. A double point cleavage is performed at the MBP-LHN/C junction and the HN-LC linker in a single incubation step with Factor Xa. The reaction is completed in a 16-hour incubation step at 22° C. with Factor Xa (NEB) at 1 U/100 μg fusion protein. The cleaved protein is diluted with 20 mM Hepes to a buffer composition of 20 mM Hepes, 25 mM NaCl, pH 7.2 and processed through a second Q Sepharose column to separate the MBP from LH_{N}/C. Activated (disulphide-bonded cleaved linker) LH_{N}/C is eluted from the Q-Sepharose column by a salt gradient (20 mM Hepes, 500 mM NaCl, 1 ìM ZnCl₂, pH 7.2) in 120-170 mM salt.

See FIG. 1 for an illustration of the purification of LHN/C.

### EXAMPLE 3—PRODUCTION OF A CONJUGATE OF IL-13 AND LH_{N}IC

Materials

SPDP is from Pierce Chemical Co.

PD-10 desalting columns are from Pharmacia.

Dimethylsulphoxide (DMSO) is kept anhydrous by storage over a molecular sieve.

Denaturing sodium dodecylsulphate polyacrylamide gel electrophoresis (SDS-PAGE) and non-denaturing polyacrylamide gel electrophoresis is performed using gels and reagents from Novex.

Additional reagents are obtained from Sigma Ltd.

LH_{N}/C is prepared according to Example 2

Human IL-13 is obtained from Sigma

Methods

Lyophilised IL-13 is rehydrated in 50 mM sodium phosphate, pH 7.5, 5 mM EDTA to a final concentration of 1 mg/ml. SATA reagent is dissolved in DMSO at a concentration of 65 mM (15 mg/ml).

To each ml of IL-13 solution is added 5 ii of the SATA solution, gently mixed, then incubated at 4° C. overnight to achieve derivatisation of the IL-13. In order to separate derivatised IL-13 from reaction components and by-products, the derivatisation mixture is applied to a PD-10 column (previously equilibrated in 50 mM sodium phosphate, pH 7.5, 1 mM EDTA).

To deprotect the acetylated —SH groups, 100 ìl of 0.5 M hydroxylamine hydrochloride in 50 mM sodium phosphate, pH 7.5, 25 mM EDTA is added to each ml of the SATA-modified IL-13 solution. These materials are mixed and reacted for 2 hours at room temperature, after which time the sulphydryl-modified IL-13 is purified by passage through a PD-10 column equilibrated in 50 mM sodium phosphate, pH 7.5, 1 mM EDTA.

The LH_{N}/C is desalted into PBS and the resulting solution (2 mg/ml) reacted with a three-fold molar excess of SPDP by addition of a 10 mM stock solution of SPDP in DMSO. After 4 h at room temperature the reaction is terminated by desalting over a PD-10 column into PBSE.

A portion of the derivatized LH_{N}/C is removed from the solution and reduced with DTT (5 mM, 30 min). This sample is analyzed spectrophotometrically at 280 nm and 343 nm to determine the degree of derivatisation. The degree of derivatisation achieved is approximately 3 mol/mol.

The bulk of the derivatized LH_{N}/C and the derivatized IL-13 are mixed in proportions such that the IL-13 is in greater than 3-fold molar excess. The conjugation reaction is allowed to proceed for >16 h at 4° C.

The product mixture is centrifuged to clear any precipitate that has developed. The supernatant is subsequently concentrated by centrifugation through concentrators (with 10000 molecular weight exclusion limit) before application to a Superose 12 column on an FPLC chromatography system (Pharmacia). The column is eluted with PBS and the elution profile followed at 280 nm.

Fractions are analyzed by SDS-PAGE on 4-20% polyacrylamide gradient gels, followed by staining with Coomassie Blue. The major conjugate products have an apparent molecular mass of between 105-115 kDa, these are separated from the bulk of the remaining unconjugated LH_{N}/C and more completely from the unconjugated IL-13 The fractions containing conjugate are pooled, dialysed against PBS, and stored at 4° C. until use.

### EXAMPLE 4—PRODUCTION OF A SINGLE POLYPEPTIDE FUSION CONJUGATE OF IL-13 AND LH_{N}IC

The methodology described below for the preparation of an IL-13-LH_{N}/C fusion is derived in part from previous studies that have described recombinant single polypeptide fusions of IL-13 (for example; the preparation of recombinant fusion of IL-13 and a truncated form of pseudomonas exotoxin (Debinski et al., 1995, J. Biol. Chem., 270, 16775-16780); the preparation of IL-13-diphtheria toxin fusions (Li et al., 2002, Prot Eng., 15, 419-427)).

Methods

The cytokine endopeptidase fusion gene is assembled using DNA fragments encoding human IL-13 (for sequence information see GenBank Accession NM_002188) spliced to LH_{N}/C with a range of short linkers introduced at the interleukin-endopeptidase junction. Within the native LH_{N}/C sequence is a specific activation site that is susceptible to cleavage by Factor Xa.

The LH_{N}/C-IL-13 fusion is expressed in E. coli under standard conditions as a maltose binding protein—LH_{N}/C—linker—IL13 fusion and soluble protein isolated using the N-terminal affinity tag. Following cleavage of the fusion with Factor Xa, activated LH_{N}/C-IL13 is isolated by ion-exchange chromatography.

### EXAMPLE 5—ACTIVITY OF IL-13-LH_{N}IC CONJUGATE IN MUCUS RELEASING CELLS

In order to confirm that IL13-LH_{N}/C is an effective inhibitor of mucus release, the effect of IL13-LH_{N}/C on release of mucins from in vitro cultures of the human colonic epithelial cell line LS180, and the normal human tracheo-bronchial epithelial (NHTBE) cell line is measured. When IL13-LH_{N}/C is applied to LS180 and NHTBE cells, there is a marked decrease in subsequent stimulated release of mucin, as measured by an ELISA specific for MUC5AC. Additionally, cleavage of syntaxin by internalised LH_{N}/C is measured to confirm that the mechanism of inhibition of secretion is via SNARE protein cleavage.

Materials

Ionomycin and ATP are obtained from Sigma

Anti-MUC5AC antisera are obtained from Neomarkers (clone 1-13M1).

Western blotting reagents were obtained from Novex & Amersham.

LS180 cells are obtained from European Collection of Animal Cell Cultures.

NHTBE cells are obtained from Clonetics.

Methods

LS180 cells are seeded onto 24 well plates and cultured in MEM-Glutamax medium (Gibco) containing 10% foetal bovine serum, 2 mM L-glutamine, 1% pen-Strep, 1% NEAA, 1% HEPES, 1% sodium bicarbonate for 3 days prior to use. IL13-LH_{N}/C is applied for 72 hours, the cells are washed to remove unbound IL13-LH_{N}/G, and the stimulated release of MUC5AC mucin assayed by ELISA.

NHTBE cells are cultured as described by Gray et al. Am. J. Respir. Cell Biol., 14, 104-112 (1996). Briefly, P2 cells are seeded into Transwell-COL collagen coated membrane supports (12 well) and cultured in bronchial epithelial cell growth medium (BEGM) for 7 days. On day 8 the media above the membrane is removed to create an air-liquid interface and the cells are cultured for a further 4 weeks by when cillia have developed. The cultures are then ready for experimental use. IL13-LH/C is applied for 72 hours, the cells are washed to remove unbound IL13-LH_{N}/C, and the stimulated release of MUC5AC mucin assayed by ELISA.

After treatment IL13-LH_{N}/C, the cells are washed three times with 1 ml/well basal salt solution (BSS). BSS, 0.5 ml/well, is then added and the cells incubated at 37° for 30 mins. The BSS is then removed to eppendorfs on ice, and replaced with BSS containing stimulant (for LS180s, 10 μM Ionomycin; for NCI-H292s, 300 μM ATP). Again the cells are incubated at 37° for 30 mins. The superfusates are then also removed to eppendorfs on ice. The cells are then lysed with 450 μl of 0.2M NaOH/well, for 10 minutes at room temp. and then neutralised with 450 μl 0.2M HCL. The cells are scraped from the plate, and the lysate removed to marked eppendorfs. The lysate is split in half and to one half, for ELISA, 50 μl HEPES added. The remaining lysate is processed for membrane protein analysis. All samples are stored at −20° C. until assay.

For the ELISA the samples are thawed at 4° C., centrifuged at 13,000×g for 10 min. and the ELISA performed. One hundred μl of supernatant is pipetted, in duplicate, from each tube to a 96 well maxisorp plate (Nunc). Fifty μl of assay buffer is used as a blank. The plate is placed in a 40° C. oven overnight, or until dry and then washed three times in PBS and blotted dry. The plate is blocked with 100 μl PBS containing 2% BSA, fraction V for 1 hour on a shaker at room temperature and then, again, washed three times in PBS and blotted dry. The plate is then incubated with 50 μl of anti MUC5AC (clone 1-13M1, Neomarkers) 1:1000, diluted in PBST (0.05% tween) for 1 hour on a shaker at room temperature, washed three times in PBS and blotted dry. One hundred μl of horseradish peroxidase anti-mouse IgG (1:2000) is added to each well, incubated for 1 hour on a shaker at room temperature, the plate washed three times in PBS and blotted dry. Two hundred μl of TMB is added to each well, colour allowed to develop, and then 50 μl of 0.5M HCl added to stop the reaction. The final colour reaction is read at 450 nm.

To the lysate for membrane protein analysis Triton-X-114 (10%, v/v) is added to extract the membrane proteins, and incubated at 4° C. for 60 min. The insoluble material is removed by centrifugation and the supernatants are warmed to 37° C. for 30 min. The resulting two phases are separated by centrifugation and the upper phase discarded. The proteins in the lower phase are precipitated with chloroform/methanol for analysis by Western blotting.

The samples are separated by SDS-PAGE and transferred to nitrocellulose. Proteolysis of syntaxin, a crucial component of the secretory process and the substrate for the zinc-dependent endopeptidase activity of BoNT/C, is then detected by probing with an anti-syntaxin antibody (clone HPC-1, Sigma) that recognises both the intact and cleaved forms of syntaxin. Cleaved syntaxin is observed.

### EXAMPLE 6—ACTIVITY OF IL13-LH_{N}/C IN AN EX VIVO MODEL OF COPD

The effect of IL13-LH_{N}/C on mucus secretion is studied in ex vivo tracheal organ bath airway models (ferret trachea). Antisera to the cleaved SNARE proteins permit immunocytochemistry for cleaved substrate proteins in the tissue samples. Cleavage of substrate proteins is correlated with blockade of stimulated mucus secretion by measurement of mucus secretion in the ex vivo trachea using Ussing chambers as described in Ramnarine et al, Br. J. Pharmacol. 113, 1183-1190 (1994). Briefly, tissue are exposed to [³⁵S]O₄ to radiolabel sulphated residues in mucus and the effects of IL13-LH_{N}/C on mucus secretion stimulated by electrical stimulation or the specific C-fibre agonist, capsaicin, are assessed

### EXAMPLE 7—IN VIVO EFFICACY OF IL13-LH_{N}/C IN REDUCING THE SYMPTOMS OF COPD

A patient, age 55, experiencing chronic obstructive pulmonary disorder is treated by intra-airway administration, for example by nebuliser, with between 0.0001 mg/kg and 1 mg/kg of an agent comprising an IL13-LH_{N} conjugate, the particular agent dose and site of injection, as well as the frequency of agent administrations depend upon a variety of factors within the skill of the treating physician, as previously set forth. Within 1-7 days after agent administration the patient's symptoms are substantially alleviated. The duration of alleviation of symptoms is from about 2 to about 6 months.

A second patient, age 63, experiencing chronic obstructive pulmonary disorder is treated by intra-airway administration, for example by nebuliser, with between 0.0001 mg/kg and 1 mg/kg of an agent comprising an IL13-LH_{N} conjugate, the particular agent dose and site of injection, as well as the frequency of agent administrations depend upon a variety of factors within the skill of the treating physician, as previously set forth. Within the first day the symptoms worsen due to excessive release of mucus, and the patient is treated with short-acting mucolytic agents (for example carbocysteine, mecysteine hydrochloride) as an inhibitor of the symptoms resulting from IL13-stimulated mucus secretion. The use of the mucolytic is stopped after 2 days. Within 3-7 days after agent administration the patient's symptoms are substantially alleviated. The duration of alleviation of symptoms is from about 2 to about 6 months.

### EXAMPLE 8—PRODUCTION OF SINGLE POLYPEPTIDE FUSION OF IL13-IGA PROTEASE

Methods

The cytokine endopeptidase fusion gene is assembled using DNA fragments encoding human IL-13 (for sequence information see GenBank Accession NM_002188) spliced to IgA protease with a range of short linkers introduced at the interleukin-protease junction. The gene encoding the IgA protease from N. gonorrhoeae is known. Primers are derived therefrom, and the gene encoding the specific protease is isolated by PCR from a nucleic acid preparation obtained from N. gonorrhoeae.

The coding region for IgA protease is inserted in frame to the 3′ end of the gene encoding IL13 and the entire cassette representing the IL13-IgA fusion is inserted in frame to the 3′ of the gene encoding maltose binding protein (MBP) in the expression-vector pMAL (New England Biolabs) to create pMAL-c2x-IL13-IgA. In this construct the maltose binding protein component can be removed from the fusion by treatment with Factor Xa protease.

pMAL-c2x-IL13-IgA is transformed into E. coli and cultured in Terrific broth complex medium in 8 L fermentor systems. Pre-induction bacterial growth are maintained at 30° C. to an OD600 nm of 8.0, at which stage expression of recMBP-IL13-IgA is induced by addition of IPTG to 0.5 mM and a reduction in temperature of culture to 25° C. After 4 hr at 25° C. the bacteria are harvested by centrifugation and the resulting paste stored at −70° C.

The cell paste is resuspended in 50 mM Hepes pH 7.2, 1 ìM ZnCl₂ at 1:6 (w/v) and cell disruption is achieved using an APV-Gaulin lab model 1000 homogeniser or a MSE Soniprep 150 sonicator. The resulting suspension is clarified by centrifugation prior to purification.

Following cell disruption and clarification, the MBP-fusion protein is isolated by ion-exchange chromatography. Cleavage of the fusion to remove the MBP purification tag is achieved by incubating with Factor Xa (NEB) at 1 U/100 ìg fusion protein for 16-hour at 22° C. The cleaved protein is separated from the free MBP by a further ion-exchange step.

### EXAMPLE 9—ASSESSMENT OF AGONIST ACTIVITY OF INSULIN

Insulin affects target cells via its interaction with the insulin receptor and the subsequent activation of downstream signalling molecules. In order to demonstrate that insulin is an agonist in the context of this invention, i.e. that insulin increases exocytic vesicle fusion, the following methods can be employed:

Firstly, presentation of GLUT4 at the plasma membrane of the cell can be monitored by immunofluorescence staining of plasma membrane sheets (as described by Fingar et al., 1993, J. Biol. Chem., 268, 3005-3008). 3T3-L1 cells are grown and differentiated on glass coverslips. Following treatment with insulin, the coverslips are washed in ice-cold buffer containing 50 mM Hepes (pH 7.4) and 100 mM NaCl. The cells are then subjected to sonication in buffer containing 20 mM Hepes (pH 7.4), 100 mM KCl, 2 mM CaCl₂, 1 mM MgCl₂, 1 μg/ml leupeptin, 10 μg/ml aprotinin and 2 mM phenylmethylsulphonyl fluoride (PMSF). The plasma membrane sheets are incubated with a rabbit antisera raised against a C-terminal GLUT4 peptide followed by a secondary incubation with a rhodamine-conjugated anti-rabbit IgG. Images are obtained by confocal microscopy. Increased flouresence due to plasma membrane localised GLUT4 is observed in membranes from insulin treated cells compared to control cells.

Secondly, the effect of presentation of GLUT4 at the plasma membrane of the cells can be monitored by assessment of enhanced glucose uptake into the 3T3-L1 adipocytes. Following 2 hour serum deprivation of adipocytes, cells are treated with insulin (100 nM) for 20 minutes, washed twice, and glucose transport assayed in HEPES-buffered saline solution (140 mM NaCl, 20 mM HEPES-Na, 2.5 mM MgSO₄, 1 mM CaCl₂, 5 mM KCl, pH 7.4) containing 10 μM 2-deoxy-D-glucose (0.5 μCi/ml 2-deoxy-D-[³H]glucose). After 10 minutes at 37° C. the reaction is stopped by aspiration of the glucose solution and rapid washing with ice cold phosphate buffered saline. Cells are lysed by the addition of 0.2M NaOH and the solution neutralised by the addition of 0.2M HCl. Uptake of [³H] 2-deoxyglucose is measured by liquid scintillation counting.

### EXAMPLE 10—EXPRESSION AND PURIFICATION OF CATALYTICALLY ACTIVE RECOMBINANT LH_{N}/B

The methodology described below will purify catalytically active LH_{N}/B protease from E. coli transformed with the appropriate plasmid encoding the LH_{N}/B polypeptide. It should be noted that various sequences of suitable LH_{N}/A and LH_{N}/B polypeptides have been described in PCT/GB97/02273, granted U.S. Pat. No. 6,461,617 and U.S. patent application Ser. No. 10/241,596, incorporated herein by reference.

Methods

The coding region for LH_{N}/B is inserted in-frame to the 3′ of the gene encoding maltose binding protein (MBP) in the expression vector pMAL (New England Biolabs) to create pMAL-c2x-LH_{N}/B. In this construct, the expressed MBP and LH_{N}/B polypeptides are separated by a Factor Xa cleavage site, and the LC and H_{N} domains are separated by a peptide that is susceptible to cleavage with enterokinase. The expression clone is termed pMAL-c2X-synLH_{N}/B.

pMAL-c2X-synLH_{N}/B is transformed into E. coli HMS174 and cultured in Terrific broth complex medium in 8 L fermentor systems. Pre-induction bacterial growth is maintained at 37° C. to an OD600 nm of 5.0, at which stage expression of recMBP-LH_{N}/B is induced by addition of IPTG to 0.5 mM and a reduction in temperature to 30° C. After four hours at 30° C. the bacteria are harvested by centrifugation and the resulting paste stored at −70° C.

The cell paste is resuspended in 20 mM Hepes pH 7.2, 125 mM NaCl, 1 ìM ZnCl₂ and cell disruption achieved using an APV-Gaulin lab model 1000 homogeniser or a MSE Soniprep 150 sonicator. The resulting suspension is clarified by centrifugation prior to purification.

Following cell disruption, the MBP-fusion protein is captured either on an amylose affinity resin in 20 mM Hepes pH 7.2, 125 mM NaCl, 1 ìM ZnCl₂, or on a Q-Sepharose FF anion-exchange resin in 50 mM Hepes pH 7.2, 1ìM ZnCl₂ with no salt. A single peak is eluted from the amylose resin in the same buffer plus 10 mM maltose and from the Q-Sepharose in 150-200 mM salt. Cleavage of the MBP-LH_{N}/B junction is completed in an 18 hours incubation step at 22° C. with Factor Xa (NEB) at 1 U/50 μg fusion protein. A substrate (MBP-LH_{N}/B) concentration of at least 4 mg/ml is desirable for efficient cleavage to take place.

The cleaved protein is diluted with 20 mM Hepes to a buffer composition of 20 mM Hepes, 25 mM NaCl, 1 ìM ZnCl₂, pH 7.2 and processed through a Q Sepharose column to separate the MBP from LH_{N}/B. The LH_{N}/B is eluted from the Q-Sepharose column with 120-170 mM salt. The linker between the light chain and H_{N} domain is then nicked by incubation with enterokinase at 1 U/100 μg of LH_{N}/B at 22° C. for 16 hours. Finally, the enterokinase is separated from the nicked LHN/B and other contaminating proteins on a Benzamidine Sepharose column, the enzyme preferentially binding to the resin over an incubation of 30 minutes at 4° C. Purified LH_{N}/B is stored at −20° C. until required. See FIG. 2 for an illustration of the purification scheme for recLH_{N}/B.

### EXAMPLE 11—PRODUCTION OF AN INSULIN-LH_{N}/B CONJUGATE

Materials

Insulin obtained from Sigma

LH_{N}/B obtained from E. coli as described in Example 10

Methods

Lyophilised human insulin is rehydrated in 50 mM sodium phosphate, pH 7.5, 5 mM EDTA to a final concentration of 10 mg/ml. SATA reagent is dissolved in DMSO at a concentration of 650 mM (150 mg/ml).

To each ml of insulin solution is added 10 ii of the SATA solution, gently mixed, then incubated at 4° C. overnight to achieve derivatisation of the insulin. In order to separate derivatised insulin from reaction components and by-products, the derivatisation mixture is applied to a PD-10 column (previously equilibrated in 50 mM sodium phosphate, pH 7.5, 1 mM EDTA).

To deprotect the acetylated —SH groups, 100 ìl of 0.5 M hydroxylamine hydrochloride in 50 mM sodium phosphate, pH 7.5, 25 mM EDTA is added to each ml of the SATA-modified insulin solution. These materials are mixed and reacted for 2 hours at room temperature, after which time the sulphydryl-modified insulin is purified by passage through a PD-10 column equilibrated in 50 mM sodium phosphate, pH 7.5, 1 mM EDTA.

The LH_{N}/B is desalted into PBS and the resulting solution (2 mg/ml) reacted with a three-fold molar excess of SPDP by addition of a 10 mM stock solution of SPDP in DMSO. After 4 h at room temperature the reaction is terminated by desalting over a PD-10 column into PBSE.

A portion of the derivatized LH_{N}/B is removed from the solution and reduced with DTT (5 mM, 30 min). This sample is analyzed spectrophotometrically at 280 nm and 343 nm to determine the degree of derivatisation. The degree of derivatisation achieved is approximately 2.5 mol/mol.

The bulk of the derivatized LH_{N}/B and the derivatized insulin are mixed in proportions such that the insulin is in greater than 3-fold molar excess. The conjugation reaction is allowed to proceed for >16 h at 4° C.

The product mixture is centrifuged to clear any precipitate that develops. The supernatant is concentrated by centrifugation through concentrators (with 10000 molecular weight exclusion limit) before application to a Superose 12 column on an FPLC chromatography system (Pharmacia). The column is eluted with PBS and the elution profile followed at 280 nm.

Fractions are analyzed by SDS-PAGE on 4-20% polyacrylamide gradient gels, followed by staining with Coomassie Blue. The major conjugate products have an apparent molecular mass of between 100-110 kDa; these are separated from the bulk of the remaining unconjugated LH_{N}/B and more completely from the unconjugated insulin.

### EXAMPLE 12—ACTIVITY OF INSULIN-LH_{N}/B IN ADIPOSE CELLS

Presentation of GLUT4 at the plasma membrane of the cell can be monitored by immunofluorescence staining of plasma membrane sheets (as described by Fingar et al., 1993, J. Biol. Chem., 268, 3005-3008). 3T3-L1 cells are grown and differentiated on glass coverslips. Following treatment with a range of concentrations of insulin or insulin-LH_{N}/B, the cells are washed twice and incubated in 8% CO₂ for 2 hours in serum free Dulbecco's modified Eagles medium, after which the cells are incubated in Krebs Ringer phosphate (with or without 100 mM insulin) for 15 minutes at 37° C. The coverslips are then washed in ice-cold buffer containing 50 mM Hepes (pH 7.4) and 100 mM NaCl. The cells are then subjected to sonication in buffer containing 20 mM Hepes (pH 7.4), 100 mM KCl, 2 mM CaCl₂, 1 mM MgCl₂, 1 μg/ml leupeptin, 10 μg/ml aprotinin and 2 mM phenymethylsulphony fluoride (PMSF). The plasma membrane sheets are incubated with a rabbit antisera raised against a C-terminal GLUT4-peptide followed by a secondary incubation with a rhodamine-conjugated anti-rabbit IgG. Images are obtained by confocal microscopy. Increased fluorescence due to plasma membrane localised GLUT4 is observed in membranes from insulin treated cells compared to control cells. In contrast, a decreased presentation of plasma membrane GLUT4 is observed in membranes from insulin-LH_{N}/B treated cells compared to controls.

Alternatively, the long term decrease in glucose uptake into adipocytes can be assessed. 3T3-L1 adipocytes are differentiated from 3T3-L1 fibroblasts by treatment with dexamethasone, 3-isobutyl-1-methylxanthine and insulin as described (Frost, S C & Lane, M D. 1985, J. Biol. Chem., 260, 2646-252). Seven days after differentiation the 3T3-L1 adipocytes are treated with a range of concentrations of the insulin-LH_{N}/B conjugate diluted into Dulbecco's modified Eagles medium. Cells are incubated for 24 to 72 hours at 37° C. in 8% CO₂. The cells are washed twice and incubated in 8% CO₂ for 2 hours in serum free Dulbecco's modified Eagles medium, after which the cells are incubated in Krebs Ringer phosphate (with or without 100 mM insulin) for 15 minutes at 37° C. Glucose uptake is initiated by the addition of [³H] 2-deoxyglucose. After 10 minutes at 37° C. the reaction is stopped by aspiration of the glucose solution and rapid washing with ice cold phosphate buffered saline. Cells are lysed by the addition of 0.2M NaOH and the solution neutralised by the addition of 0.2M HCl. Uptake of [³H] 2-deoxyglucose is measured by liquid scintillation counting.

### EXAMPLE 13—IN VIVO EFFICACY OF INSULIN-LH_{N}/B IN REDUCING THE SYMPTOMS OF OBESITY

A patient, age 34, experiencing chronic obesity is treated by administration of between 0.0001 mg/kg and 1 mg/kg of an agent comprising an insulin-LH_{N}/B conjugate, the particular agent dose and site of injection, as well as the frequency of agent administrations depend upon a variety of factors within the skill of the treating physician, as previously set forth. When coupled with an appropriate low glucose diet, the patient's symptoms are substantially alleviated 4 weeks post administration. The duration of alleviation of symptoms is from about 2 to about 6 months.

### EXAMPLE 14—ASSESSMENT OF THE AGONIST ACTIVITY OF MAST CELL DEGRANULATING PEPTIDE (MCD PEPTIDE)

The ability of mast cell degranulating (MCD) peptide to initiate release of inflammatory mediators from mast cells is well documented (see Baku review article; 1999, Peptides, 20, 415-420). For this reason, experimental assessment of agonist properties of MCD peptide is not required.

### EXAMPLE 15—PRODUCTION OF A SINGLE POLYPEPTIDE FUSION OF MCD PEPTIDE AND LH_{N}/IC

Methods

The peptide endopeptidase fusion gene are assembled using DNA fragments encoding human MCD peptide (for sequence information see Baku, 1999, Peptides, 20, 415-420 or GenBank Accession S78459) spliced to the 3′ end of DNA encoding the LH_{N}/C polypeptide. A range of short linkers are introduced at the MCD peptide-endopeptidase junction. Within the native LH_{N}/C sequence is a specific activation site that is susceptible to cleavage by Factor Xa.

The LH_{N}/C-MCD peptide fusion is expressed in E. coli under standard conditions as a maltose binding protein—LH_{N}/C—linker—MCD fusion and soluble protein isolated using the N-terminal affinity tag. Following cleavage of the fusion with Factor Xa, activated LH_{N}/C-MCD peptide is isolated by ion-exchange chromatography.

### EXAMPLE 16—ACTIVITY OF MCD PEPTIDE-LH_{N}/C IN MAST CELLS

Mast cells are obtained by peritoneal lavage of large (>300 g) male Sprague Dawley rats. The mast cells are isolated from contaminating cells types by centrifugation through a cushion of Percoll. They are washed twice by resuspension and centrifugation and finally suspended in an iso-osmotic buffered salt solution (290 mOsm) which comprises NaCl (137 mM), KCl (2.7 mM), MgCl₂ (2 mM), PIPES (20 mM), BSA (1 mg·ml⁻¹), pH 6.8. The cells are incubated with MCD peptide-LH_{N}/C at 37° C. for 16 hours, are washed twice by resuspension and centrifugation, and then suspended at approximately 3×10⁵ cells ml¹′ in buffered salt solution. The cells are transferred to the wells of a 96-Vwell microtitre plate. Mast cells are stimulated to degranulate by IgE cross-linking. Purified mast cells, 90 micolitre per well, are challenged for 2 hours at 37° C. with anti-IgE (3 microgm ml⁻¹). After incubation the reaction is quenched by the addition of 100 microlitre of ice cold buffer and the cells are sedimented by centrifugation (5 min, 400 g, at 4° C.). Samples (50 microlitre) of supernatant are transferred to equivalent wells in black plastic, opaque microtitre plates for analysis of secreted 3-D-N-acetylglucosaminidase (hexosaminidase). The reaction is initiated by the addition of 50 microlitre of a solution of 4-methylumbelliferyl-acetyl-3-D glucosaminide (1 mM in Na citrate, 200 mM, pH 4.5, containing Triton X100, 0.01%). After incubation at 37° C. for about 3 hours, the reaction is terminated by the addition of 150 microlitre of TRIS (0.2 M). Fluorescence (355-460 nm) is measured on a microtitre plate reader. Calculation of % secretion is based on comparison of fluorescence measured with no cells and the total cell hexominidase content as released by Triton X-100 (0.1%).

### EXAMPLE 17—IN VIVO EFFICACY OF MCD PEPTIDE-LH_{N}IC IN REDUCING THE SYMPTOMS OF ASTHMA

A patient, age 35, experiencing asthma is treated by intra-airway administration, for example by nebuliser, with between 0.0001 mg/kg and 1 mg/kg of an agent comprising a MCD peptide-LH_{N}/C conjugate, the particular agent dose and site of injection, as well as the frequency of agent administrations depend upon a variety of factors within the skill of the treating physician, as previously set forth. Within 1-7 days after agent administration the patient's symptoms are substantially alleviated. To alleviate short-term increase in the severity of symptoms experienced by the patient following administration of the agent, the mast cell stabiliser disodium cromoglycate is administered. The duration of alleviation of symptoms is from about 2 to about 6 months.

### EXAMPLE 18—ASSESSMENT OF IL4 AGONIST ACTIVITY

In order to confirm that IL4 is an agonist, i.e. that IL4 increases exocytic fusion in a target cell, the effect of IL4 on membrane presentation of CD23 (the low affinity IgE receptor) is measured.

Materials

Human IL4 was obtained from Sigma

Methods

The effect of IL4 on the expression of B-cell surface antigens such as CD23 is investigated by flow cytometry. Incubation of human monocytes for 48 hours in the presence of 30 U/ml IL4 results in strong induction of CD23 expression, as identified by Flow cytometry using anti-CD23 monoclonal antibodies (Becton Dickenson).

### EXAMPLE 19—PRODUCTION OF A SINGLE POLYPEPTIDE FUSION OF IL4-LHIC

The methodology described below for the preparation of an IL4-LH_{N}/C fusion is similar to previously described for IL13-LH_{N}/C

Methods

The cytokine endopeptidase fusion gene is assembled using DNA fragments encoding human IL-4 (for sequence information see GenBank Accession AF395008) spliced to LH_{N}/C with a range of short linkers introduced at the interleukin-endopeptidase junction. Within the native LH_{N}/C sequence is a soecific activation site that is susceptible to cleavage by Factor Xa.

The LHN/C-IL-4 fusion is expressed in E. coli under standard conditions as a maltose binding protein—LH_{N}/C—linker—IL4 fusion and soluble protein isolated using the N-terminal affinity tag. Following cleavage of the fusion with Factor Xa, activated LH_{N}/C-IL4 is isolated by ion-exchange chromatography.

### EXAMPLE 20—ACTIVITY OF IL4-LH_{N}/C IN PREVENTING SURFACE EXPRESSION OF THE IGE RECEPTOR CD23 IN HUMAN MONOCYTES

In order to confirm that IL4-LH_{N}/C is an effective inhibitor of CD23 expression on the surface of human monocytes, membrane presentation of CD23 (the low affinity IgE receptor) is measured.

Methods

The effect of an IL4-LH_{N}/C conjugate on the expression of CD23 is investigated by flow cytometry. Human monocytes are incubated for 48 hours in the presence of IL4-LH_{N}/C. Subsequent stimulation with 30 U/ml IL4 results in strong reduction of CD23 expression, as identified by Flow cytometry using anti-CD23 monoclonal antibodies (Becton Dickenson), in the conjugate treated monocytes compared to untreated controls.

### EXAMPLE 21—ASSESSMENT OF TNFα AGONIST ACTIVITY

In order to confirm that TNF alpha (TNFα) is an agonist, the effects of the proinflammatory cytokine on the release of soluble E-selectin and P-selectin and vascular cell adhesion molecule 1 (VCAM-1) expression, are investigated using synovial microvascular endothelial cells (SMEC) and macro vascular human umbilical vein endothelial cells (HUVE).

Stimulation of VCAM and P-selectin expression and release of E-selection TNFα stimulated endothelial cells demonstrates the agonist activity of TNFα.

Materials

Anti-rat E, P-selectin and VCAM-1 was obtained from Sigma

Rat TNFα_ was obtained from Sigma

ELISA materials for assessment of release E-selectin were obtained from Biocarta US

Methods

Cultured endothelial cells (HUVE and SMEC) are treated for 4 hours with medium alone or TNFα_. The expression of selectin and endothelial adhesion molecules (VCAM) is evaluated by flow cytometry (as described by Polgar et al., 2002, Blood, 100(3), 1081-3). Whilst release of E-selection is measured by ELISA (following methodology supplied by manufacturer) of the supernatant removed from the cells.

### EXAMPLE 22—IN VIVO EFFICACY OF TNFα-LH_{N}/C IN REDUCING THE SYMPTOMS OF INFLAMMATION

In vitro studies show that TNFα is a critical and proximal mediator of the inflammatory pathway in the rheumatoid joint. TNFα-LH_{N}/C dramatically reduces inflammation and slows or halts the structural damage in both early treatment in the onset of disease and at later stages. In human terms, these efficacies translate to less functional disability and higher quality of life.

A 56 year old patient presenting with an RA condition is treated with between 0.0001 mg/kg and 1 mg/kg of an agent comprising an TNFα-LH_{N}/C conjugate. This agent prevents vesicular release of P-selectin, leading to a marked reduction of symptoms of pain, stiffness, swelling and tenderness of joints within 24 hours. Maximum benefits are observed for around 2-4 months.

The response to treatment with TNFα-LH_{N}/C in rheumatoid arthritis (RA) and inflammatory bowel disease are likely to be repeated in any chronic (non-infectious) inflammatory disease that is primarily macrophage-driven, for example Wegener's granulomatosis, psoriatic arthritis and congestive heart failure.

### EXAMPLE 23—ASSESSMENT OF AGONIST ACTIVITY OF INSULIN INCREASING PRESENTATION OF NMDA CHANNELS IN HIPPOCAMPAL AND CEREBRAL CORTEX NEURONS

Insulin, insulin receptors, and their substrates are enriched at synapses in hippocampus and cerebral cortex where they are thought to perform a number of functions including regulation of glucose metabolism, gene expression, and synaptic plasticity.

Using a variety of methods Skeberdis et al (Proc. Natl. Acad. Sci., 2001, 98(6), 3561-3566) have demonstrated that insulin treatment results in the delivery of new NMDA channels to the plasma membrane by regulated exocytosis, i.e. insulin increases exocytic fusion. This has been confirmed by demonstrating a reduction in insulin-induced delivery of NMDA channels to the cell surface following cleavage of SNAP-25. Though described fully in the literature, methods to confirm the agonist activity of insulin in relation to channel presentation are reproduced here to aid understanding.

Firstly, insulin potentiation of activity of recombinant NMDA expressed in Xenopus oocytes is investigated by electrophysiology. Adult female Xenopus laevis (Xenopus I, Ann Arbor Mich.) are maintained in a temperature- and light-controlled environment and injected with in vitro-transcribed mRNAs (20 ng mRNA/cell) encoding subunits of the NMDA channel. Whole-cell currents are recorded from oocytes (2-6 days after injection) at ambient temperature in the voltage clamp mode as described (Zheng, X., Zhang, L., Wang, A. P., Bennett, M. V. L. & Zukin, R. S. (1997) J. Neurosci. 17, 8676-8686). Recordings show insulin potentiates NMDA-channel dependent currents by a mechanism that involves increased channel presentation rather than NMDA channel modification.

The patch clamp recordings are supplemented by a Western blot analysis of NMDA channel presentation. Using an antibody specific for the NR1 subunit of NMDA channels, and a surface protein biotinylation protocol (described by to Chen, N., Luo, T. & Raymond, L. A. (1999) J. Neurosci. 19, 6844-6854) enhanced expression of channels is observed.

### EXAMPLE 24—PRODUCTION OF A CONJUGATE FOR DELIVERY OF DNA ENCODING LCIC INTO A CELL

According to the methodology described by Cotton et al (Cotton, M., Wagner, E. and Birnstiel, L. (1993) Receptor-mediated transport of DNA into eukaryotic cells. Methods in Enzymol. 217, 619-645) and others, DNA encoding a protein of interest can be transfected into eukaryotic cells through receptor-mediated endocytosis of a protein-DNA conjugate. Several methods exist for condensing DNA to a suitable size using polycationic ligands. These include: polylysine, various cationic peptides and cationic liposomes. Of these, polylysine was used in the present study because of its successfully reported use in receptor-mediated transfection studies (Cotton et al., 1993). Using such an approach, the construction of an IL13-H_{N}-[LC/C] conjugate is described below, where [LC/C] represents the polylysine condensed DNA encoding the light chain of botulinum neurotoxin type C.

Materials

SPDP is from Pierce Chemical Co.

Additional reagents are obtained from Sigma Ltd.

Methods

The methodology described below for the preparation of an IL-13-H_{N}/C fusion is derived in part from previous studies that have described recombinant single polypeptide fusions of IL-13 (for example; the preparation of recombinant fusion of IL-13 and a truncated form of pseudomonas exotoxin (Debinski et al., 1995, J. Biol. Chem., 270, 16775-16780); the preparation of IL-13-diphtheria toxin fusions (Li et al., 2002, Prot Eng., 15, 419-427)). The cytokine-H_{N}/C fusion gene is assembled using DNA fragments encoding human IL-13 (for sequence information see GenBank Accession NM_002188) spliced to the H_{N} domain of BoNT/C with a range of short linkers introduced at the interleukin-translocation domain junction to facilitate correct folding.

Alternatively, the H_{N}-IL-13 fusion gene is derived by polymerase chain reaction from the LH_{N}/C-IL-13 construct described in Example 4. The fusion derived by either method is expressed in E. coli under standard conditions as a maltose binding protein—H_{N}-linker—IL13 fusion and soluble protein isolated using the N-terminal affinity tag. Following cleavage of the fusion with Factor Xa, H_{N}—IL13 is isolated by ion-exchange chromatography. Using a plasmid containing the gene encoding LC/C under the control of the CMV (immediate early) promoter, condensation of DNA was achieved using SPDP-derivatised polylysine to a ratio of 2 DNA to 1 polylysine. Conjugates were then prepared by mixing condensed DNA (0.4 mg/ml) with H_{N}-IL-13 (100 μg/ml) for 16 hr at 25° C. The SPDP-derivatised polylysine and the free —SH group present on the H_{N} domain combine to facilitate covalent attachment of the DNA and protein.

It will be appreciated by one skilled in the art that similar methods for producing agonist-H_{N} fusions could be employed for other agonists as exemplified in this patent.

### EXAMPLE 25—PRODUCTION OF SINGLE POLYPEPTIDE FUSION CONJUGATE OF EGF AND LH_{N}/C

Epidermal Growth Factor (EGF) was identified, in accordance with the present invention, as a potential agonist of mucin release. In more detail, EGF was identified by way of a literature review—Perrais, M. et al (2002) J. Biol. Chem., August 30, 277(35), pp. 32258-67; and Takeyama, K. et al (1999) proc. Natl. Acad. Sci. USA, March 16, 96(6), pp. 3081-6. The agonist activity of EGF was confirmed by said literature, and also by Example 27.

Method

An endopeptidase fusion gene is assembled using DNA fragments encoding human EGF spliced to LHN/C with a range of short linkers introduced at the endopeptidase-growth factor junction. Within the native LHN/C sequence is a specific activation site that is susceptible to cleavage by Factor Xa.

Expression of the fusion is performed using standard expression conditions. An overnight culture is prepared by the addition of a microbank bead to 100 ml Terrific Broth plus 100 μg/ml ampicillin, 37 μg/ml chioramphenicol, and culture performed at 37° C., 225 RPM overnight. 100 ml of the overnight culture is used to inoculate 1 L Terrific Broth plus 100 μg/ml ampicillin, 37 μg/ml chloramphenicol, 0.5% glucose. The culture is incubated at 30° C. until OD600 reaches ˜0.6, at which stage the temperature is lowered to 16° C. and the culture cooled for −1 hour. Expression of the fusion is induced by addition of IPTG to 1 mM, followed by incubation of the culture overnight at 16° C. The culture is centrifuged at 4500 rpm for 20 mins in a RC3BP centrifuge with a H6000A rotor. The cell paste is resuspended in 50 mM Hepes pH 8.0 and stored-at −20 (C prior to purification. Purification is achieved using a combination of two affinity matrices. The following buffers are prepared in advance:

Buffer A: 50 mM Hepes pH8.0, 200 mM NaCl

Buffer B: 50 mM Hepes pH8.0, 200 mM NaCl, 20 mM Maltose

Buffer C: 50 mM Hepes pH8.0, 25 mM NaCl

Buffer D: 50 mM Hepes pH8.0, 500 mM NaCl, 500 mM Imidazole

The cell pellet from a 1 litre culture is resuspended in −50 ml Buffer A, and PMSF added to 1 mM. Cells are disrupted by homogenisation (2 passes at 300-400 bar pressure) or sonication (6×30s pulses). The disrupted cell paste is centrifuged at 13K in an F16-250 rotor (25,560 g), or at 4000 RPM for 60 mins in a megafuge benchtop centrifuge. The supernatant is loaded onto a 20 ml amylose column at 5 ml/min and eluted in 100% Buffer B at same flow rate. 5 ml fractions are collected, pooled, and diluted to A280 ˜0.5 using Buffer A. Factor Xa is added to 1 U Fxa/100 μg protein and CaCl₂ to 1 mM. The sample is incubated overnight at 30° C. until cleavage is complete.

The cleavage reaction pool is diluted ¼ using buffer C and loaded onto a previously equilibrated 40 ml Cu²⁺ charged chelating column at 5 ml/min in Buffer C. Bound material is eluted at 5 ml/min using 10% Buffer D. 2.5 ml fractions are collected, pooled and dialysed into buffer C overnight.

The dialysed pool is loaded onto a 30 ml amylose column at 2 ml/min and the flow through is collected. Bound MBP can be eluted off the column using 100% Buffer B. The flow-through is concentrated and dialysed into 50 mM Hepes pH7.4 prior to use.

As an alternative to loading the material onto the amylose column, a 20 ml Q-sepharose fast flow column may be used. In this case, the column is equilibrated using buffer C, and the dialysed pool is loaded at 5 ml/min. The column is then eluted using 50 mM Hepes pH 8.0, 1 M NaCl at 25% and 50%. Fractions are collected, pooled and stored at −20° C.

### EXAMPLE 26—ACTIVITY OF EGF-LH_{N}/C FUSION CONJUGATE IN MUCUS RELEASING CELLS

Dose-dependent cleavage of the syntaxin SNARE protein was detected in cells treated for 3 days with EGF-LHN/C using Western blot techniques and an antibody specific to the smaller cleavage fragment of syntaxin (anti-AVKY)

Method

EGF-LHN/C is applied to the cells for 3 days in serum-free medium supplemented with L-Glutamine at a maximum concentration of 150 μg/ml. The cells are incubated at 37° C., 5% CO₂. Following treatment for 3 days the cells are lysed in 0.1 M NaOH (10 minutes at room temperature) and 0.1 M HCl and 100 μM HEPES, the lysate is solubilised with Triton-X 114 and chilled at 4° C. for 5 minutes. The lysate is then spun at 13,000 rpm in an Eppendorf microcentrifuge at 4° C. for 10 minutes. Any cloudiness in the recovered supernatant is removed by further centrifugation at 13,000 rpm at room temperature. The upper phase is then discarded and ethanol, chloroform and water are added to the supernatant in the ratio of 4:2:3. The solution is mixed by vortex and spun at 13,000 rpm for 10 minutes at room temperature. The Upper phase is discarded and the lower phase washed by the addition of methanol and further centrifugation for 10 minutes at room temperature and 13,000 rpm. The supernatant is discarded and the pellet allowed to air dry for an hour before the protein sample is analysed by SDS-PAGE and Western blotting. Western blot analysis of the cell lysates shows an increase in syntaxin protein cleavage when compared to cells treated with the LHn/C fragment alone. A fusion protein dose-dependent cleavage of syntaxin can also be demonstrated.

### EXAMPLE 27—ASSESSMENT OF AGONIST ACTIVITY OF EGF BY ASSESSING MUCIN RELEASE FROM NCI-H292 CELLS

Method

NCI-H292 cells (a mucin secreting cell line, which is publically available from the ECACC Depositary—eg. Accession No. 91091815) are seeded onto 24 well plates and fed using RPMI medium supplemented with 5% Foetal Calf Serum and 5 mM L-Glutamine. The following day cells are with 30 μg/ml of EGF in serum-free medium and incubated for 3 days at 37° C. and 5% CO₂ atmosphere. The media is collected, centrifuged at 13,000 g in a microcentrifuge at 4° C. for 5 minutes and the supernatant collected. Equal aliquots of the supernatant are added in duplicate to a Maxisorp™ ELISA plate and incubated overnight at 400 C. The plate is washed three times in PBS, blotted dry and then incubated for an hour on a plate shaker at room temperature in PBS-Tween™ 20 0.05% and anti-MUC4AC antibody (clone 1-13M1 Neomarkers) at 1/1000 dilution. The plate is washed three times in PBS, blotted dry and incubated for an hour on a plate shaker at room temperature in PBS-Tween™ 20 0.05% and anti-Mouse Horseradish peroxidase conjugated antibody at 1/2000 dilution. The plate is then washed three times in PBS and equal volumes of TMB are added to all wells and the colour allowed to develop. The reaction is stopped using 0.5 M HCl and the resulting plate read at 450 nm in a plate reader.

Results

Three micrograms/ml of EGF over a three day period causes an increase in mucin released into the medium when analysed by ELISA.

The embodiments of the disclosure in which an exclusive property or privilege is claimed are defined as follows:

1. A method of preparing a fusion protein for the inhibition or reduction of secretion of an extracellular mediator from a target cell, said method comprising:
(a) selecting a target cell that secretes a polypeptide extracellular mediator associated with a medical condition or disease;
(b) identifying a polypeptide agonist that increases exocytic fusion and secretion of said extracellular mediator from said target cell upon binding of the polypeptide agonist to a receptor on said target cell to form a polypeptide agonist-receptor complex, wherein said polypeptide agonist-receptor complex undergoes endocytosis to be incorporated into an endosome within said target cell; and
(c) preparing by recombinant expression said fusion protein, said fusion protein comprising:
(i) the polypeptide agonist identified by step (b);
(ii) a clostridial neurotoxin protease; and
(iii) a clostridial neurotoxin translocation domain;
wherein said fusion protein inhibits or reduces the secretion of said polypeptide extracellular mediator.

2. The method of claim 1, wherein step (a) comprises:
confirming said polypeptide agonist molecule is an agonist by identifying an increase in secretion of said extracellular mediator from the target cell following binding of the polypeptide agonist to the target cell.
